(19) United States
(12) Patent Application Publication
(10) Pub. No.: US 2005/0208539 A1
(43) Pub. Date: Sep. 22, 2005

(54) Quantitative amplification and detection of small numbers of target polynucleotides

(76) Inventors: Charles S. Vann, Burlingame, CA (US); Kai Qin Lao, Pleasanton, CA (US); Mark Reed, Menlo Park, CA (US); Seth R. Stern, Palo Alto, CA (US); Dennis Lehto, Santa Clara, CA (US)

Correspondence Address: HELLER EHRMAN LLP, 275 MIDDLEFIELD ROAD, MENLO PARK, CA, 94025-3506, US

(21) Appl. No.: 11/026,924

(22) Filed: Dec. 30, 2004

Related U.S. Application Data
(60) Provisional application No. 60/534,095, filed on Dec. 31, 2003.

(51) Int. Cl. C12Q 1/68; C12M 1/34

(52) U.S. Cl. 435/6; 435/287.2

(57) ABSTRACT

Devices, assemblies, systems and methods described herein enable detection of as few as a single copy of a target nucleic acid molecule. Polynucleotides copied from a single or a small number of target nucleotide(s) within regions near to an initial copying site may be detected by optical or other methods as disclosed herein. Devices, assemblies and systems may comprise probes and/or primer molecules. Systems comprising optical assemblies, thermal assemblies and reaction assemblies (having reaction chambers for amplification of target nucleic acid molecules) are provided in which used reaction assemblies may be replaced to provide reusable devices. Systems comprising analytical assemblies and detection assemblies are provided in which an assay cartridge having assay chambers may engage a thermal assembly for amplification of target nucleic acid molecules. These devices, systems and methods offer the advantages of detection of as few as a single copy of a target nucleic acid molecule.

### CROSS-REFERENCE TO RELATED APPLICATIONS

This application claims the benefit under 35 U.S.C. § 119(e) of U.S. Provisional application Ser. No. 60/534,095 filed Dec. 31, 2003, the contents of which application is hereby incorporated by reference in its entirety.

### BACKGROUND

### Field of the Invention

This invention pertains to amplification and/or detection of target nucleic acid molecules. More particularly, this invention provides systems, devices and methods suitable for the amplification and detection of a small number of copies, or even a single copy, of a target nucleic acid molecule.

There is a need for methods and devices for detecting and amplifying extremely small amounts of nucleic acid target material while minimizing interference by other, non-target, nucleic acid material.

### SUMMARY

The present invention provides methods and devices for quantitative counting of a small number of target nucleic acid molecules. For example, the small number of target nucleic acid molecules may be hundreds of copies, or tens of copies, less than about ten copies, or less than about 5 copies, and may be only one copy. In some embodiments, methods herein comprise real-time PCR methods such as Taqman® methods and assays to detect the presence and amplification of target molecules.

Devices having pathways, substrates, barriers, or other elements configured to reduce the dispersion of amplified polynucleotides from a site of amplification may be used to provide small regions containing polynucleotides copied from a single or a small number of target polynucleotide(s). Such small regions of copies of target polynucleotides may be detected by optical or other methods. Polynucleotides comprising copies of a single or a small number of target polynucleotide(s) may be detected, and may be recovered from such regions for analysis, sequencing, or further amplification or processing.

In some embodiments, a device is provided for amplifying and/or detecting a small number of nucleic acid molecules in a sample, the device having a first well region configured to receive probe molecules and primer molecules, a second well region configured to receive target nucleic acid molecules, and a connecting region between the first well region and the second well region. The probe molecules and primer molecules may each be configured to hybridize with at least a portion of a target nucleic acid molecule. The connecting region provides a restricted pathway between the well regions, allowing contact between probe molecules, primer molecules and target nucleic acid molecules, so that a small number of target nucleic acid molecules may be amplified and/or detected. A restricted pathway may be, for example, a capillary with a small bore, a shallow groove with a small cross-section, a pathway containing material that slows or occludes fluid or solute flow, a long or tortuous pathway, or other pathway that restricts the passage of material such as a fluid or a material dissolved or suspended in the fluid. For example, the small number of copies of target polynucleotides may be less than about twenty copies, may be less than about ten copies, or may be less than about 5 copies, and may be only one copy of the target polynucleotide.

In some embodiments, a device for amplifying and/or detecting a small number of polynucleotides in a sample has a well with an interior region including a well base. The device is configured to receive probe molecules, primer molecules, and target nucleic acid molecules. The well base has more than one base location, and comprises a thin gel layer that is configured to accept probe, primer and target molecules so that at least some of the probe, primer and target molecules may diffuse into said thin gel layer. Target molecules that have diffused into the thin gel layer are dispersed to different locations so that at a given location within the thin gel layer as few as a small number of target nucleic acid molecules may be contacted by probe molecules and primer molecules. In this way, a small number of target molecules may be amplified and/or detected at a given location by action of the probe and primers. In some embodiments, target nucleic acid molecules may be dispersed within a thin gel layer at a density of less than about ten target nucleic acid molecules per square micron (as viewed from above), or less than about one target nucleic acid molecule per square micron, or less than about 10⁻¹ target nucleic acid molecules per square micron, or less than about 10⁻² target nucleic acid molecules per square micron, or less than about 10⁻³ target nucleic acid molecules per square micron, or less than about 10⁻⁴ target nucleic acid molecules per square micron, or less than about 10⁻⁵ target nucleic acid molecules per square micron, or less than about 10⁻⁶ target nucleic acid molecules per square micron.

In some embodiments, a device for amplifying and/or detecting a small number of nucleic acid molecules in a sample has a well with an interior volume including a gel. The well is configured to receive probe molecules, primer molecules, and target nucleic acid molecules, and the gel is configured so that at least some of the probe, primer and target molecules may diffuse into the gel and/or be dispersed in the gel. In some embodiments, probe and/or primer molecules are dispersed in the gel before application of target to gel. Target molecules diffused into the gel disperse to different locations within the interior volume so that at a given location as few as a small number of target nucleic acid molecules may be contacted by probe molecules and primer molecules. In this way, as few as a small number of target molecules may be amplified and/or detected at a given location by action of the probe and primers. In some embodiments, target nucleic acid molecules may be dispersed within a gel volume at a density of less than about ten target nucleic acid molecules per cubic micron, or less than about one target nucleic acid molecule per cubic micron, or less than about 10⁻¹ target nucleic acid molecules per cubic micron, or less than about 10⁻² target nucleic acid molecules per cubic micron, or less than about 10⁻³ target nucleic acid molecules per cubic micron, or less than about 10⁻⁴ target nucleic acid molecules per cubic micron, or less than about 10⁻⁵ target nucleic acid molecules per cubic micron, or less than about 10⁻⁶ target nucleic acid molecules per cubic micron.

In some embodiments, a device for amplifying and/or detecting a small number of nucleic acid molecules in a sample comprises multiple hydrophilic wells separated from each other by a hydrophobic surface. The wells have an interior volume configured to receive probe molecules, primer molecules, and target nucleic acid molecules, and are sized and spaced to receive as few as a small number of target nucleic acid molecules. Contact between the small number of target molecules and probe and primer molecules within a single well is such that as few as a small number of target molecules may be amplified and/or detected within a single well.

In some embodiments, a device for amplifying and/or detecting a small number of nucleic acid molecules in a sample has a substantially planar hydrophobic surface with a plurality of walls disposed substantially perpendicular to that surface. The walls define multiple wells, and define an opening opposite the substantially planar hydrophobic surface. The wells comprise one or more depressions in the planar hydrophobic surface, the depressions having a hydrophilic surface that defines a small volume configured to receive probe molecules, primer molecules, and target nucleic acid molecules. The probe and primer molecules are each configured to hybridize with at least a portion of a target nucleic acid molecule so that as few as a small number of the target molecules may be amplified and/or detected within a depression.

In some embodiments, systems including devices for amplification and/or detection of small numbers of target nucleic acid molecules are provided. Systems described herein comprise such devices and at least one other element, or assemblies such as an optical assembly, a reaction assembly, and an observation assembly, or an analytical assembly and a detection assembly. A system may comprise a detector for detecting copies of target nucleic acid molecules or for detecting the amplification (e.g., PCR amplification) of a single or a small number of target nucleic acid molecules. A system may comprise mechanical means for holding, transporting, and otherwise manipulating a device, or a detector, or other elements of the system. Detection may be by eye and/or by a detector. A detector may comprise an optical detector, such as a video camera, a charge-coupled device, or other instrument capable of an detecting an image or an optical signal such as fluorescence. Lenses, filters, mirrors, and other optical elements may also be comprised in systems. A system may also comprise a light source, or other illumination element. A system may comprise a controller, which may comprise a computer, for controlling the operation of a system, and for coordinating the operation of the various elements of the system. A system may comprise a fluid delivery system, such as a dispenser for delivering solutions to a device. A system may comprise a fluid collection system for removing liquids.

In some embodiments, devices and systems comprising component assemblies that are operably connected together to form operable assemblages are provided. Such devices may comprise, for example, optical assemblies having, e.g., a light source a lens, a filter, or other optical elements; thermal assemblies having, e.g., a heat source, a cooling element, or thermal element; and disk assemblies having, e.g., a reaction chamber configured to react target nucleic acid molecules with reagents. Such reagents may be prepositioned (e.g., pre-dried) in a portion or portions of a component assembly prior to addition of a sample comprising a target nucleic acid molecule. In embodiments, such devices may be dis-assembled into separate assemblies after use. In embodiments, a (one or more) component assembly may be replaced by a different assembly after use for re-use in a re-assembled device comprising the replacement assembly or assemblies. Such replacement assemblies are typically the same or a similar type of component assembly as the assembly that has been removed (e.g., a used reaction assembly may be replaced by a fresh, unused reaction assembly).

Also provided are methods for amplifying and/or detecting small numbers of nucleic acid molecules. In some embodiments, a method for amplifying and/or detecting small numbers of nucleic acid molecules comprises contacting a channel with a first solution containing a primer molecule and a probe molecule. The channel is configured to conduct a solution along the channel. The solution may be conducted along a channel by capillary action, by pressure, by suction, or by other means or combination of means. The first solution is then dried, so that the primer and probe molecules are retained within the channel. A second solution containing one or more target nucleic acid molecules is contacted with the channel, which conducts the second solution along the channel, effective to contact the target nucleic acid molecule, dried probe and primer molecules with the second solution to form a target-primer-probe mixture. In some embodiments, the channel is configured to enhance separation between target nucleic acid molecules in a solution. The mixture is then thermocycled to cause amplification of at least one target nucleic acid. In related methods, the order of application of the solutions is altered, for example, the order may be reversed, so that the first solution to be applied is a solution containing one or more target nucleic acid molecules, and the second solution to be applied is a solution containing a primer molecule and a probe molecule, and the mixture is then thermocycled to cause amplification of at least one target nucleic acid.

In some embodiments, methods are provided for amplifying and/or detecting small numbers of nucleic acid molecules, including steps of contacting a solution that comprises a primer molecule and a probe molecule with a gel within a well. The probe and the primer molecules are each configured to hybridize with at least a portion of a target nucleic acid molecule. At least some of the primer and probe molecules diffuse into the gel, or are allowed to diffuse into the gel. A solution including at least one target nucleic acid molecule is contacted with the gel, mixing the target nucleic acid molecule with probe and primer molecules within the gel. The mixture is thermocycled to cause amplification of at least one target nucleic acid. Thermocycles may be repeated a number of times to produce nucleic acid copies of said target nucleic acid molecule effective to amplify and/or detect said target nucleic acid molecule. In some embodiments, the number of times the thermal cycles may be repeated may be less than about fifty times, or less than about thirty times, or less than about twenty times, or less than about ten times. In some embodiments, the number of times the thermal cycles may be repeated may be between about thirty and about fifty times, or between about twenty and about thirty times, or between about ten and about twenty times.

Also provided are methods for amplifying and/or detecting small numbers of nucleic acid molecules comprises placing a hydrophilic well in contact with a solution, removing the hydrophilic well from contact with the solution, placing at least a portion of the device in contact with a hydrophobic liquid, making copies of said target nucleic acid molecule within said solution in contact with a well; and detecting the presence of said target nucleic acid molecule or copies thereof. The hydrophilic well forms at least a portion of a device that has a hydrophilic well surrounded by a hydrophobic surface. As before, the solution comprises a target nucleic acid molecule, a primer molecule and a probe molecule, the probe molecules and primer molecules each being configured to hybridize with at least a portion of the target nucleic acid molecule. When the well is removed from contact with the solution, a portion of the solution remains in contact with the hydrophilic well, although substantially no solution remains in contact with the hydrophobic surface.

Methods of making copies herein may comprise steps of applying heat, to raise the temperature of the mixed target nucleic acid, probe and primer molecules, and of allowing the temperature to become reduced, these steps comprising a thermal cycle. These thermal cycles may be repeated a number of times. In some embodiments, the number of times the thermal cycles may be repeated may be less than about fifty times, or less than about thirty times, or less than about twenty times, or less than about ten times. In some embodiments, the number of times the thermal cycles may be repeated may be between about thirty and about fifty times, or between about twenty and about thirty times, or between about ten and about twenty times.

Also provided are methods for amplifying and/or detecting small numbers of nucleic acid molecules comprising steps of placing a portion of a substrate comprising a hydrophilic well surrounded by a hydrophobic surface in contact with a first solution including a primer and a probe molecule. The probes and primers are each configured to hybridize with at least a portion of a target nucleic acid molecule. The substrate is then dried to dry the probe and primer molecules into at least a portion of the hydrophilic well. A second solution containing a target nucleic acid molecule is then placed in contact with the hydrophilic well, and then excess second solution is removed, so that a mixture of target nucleic acid molecules, probes and primers remains in contact with the well. At least a portion of the substrate is placed into contact with a hydrophobic liquid, so that the mixture remains in contact with the well; copies of the target nucleic acid molecule are made within or adjacent the mixture, so that the presence of the target nucleic acid molecules or its copies can be detected. In related methods for amplifying and/or detecting small numbers of nucleic acid molecules, the order of application may be altered. For example, the order of application may be reversed, so that a solution containing a target nucleic acid molecule is first placed in contact with a hydrophilic well, and then a solution including a primer and a probe molecule is placed in contact with a hydrophilic well, so that a mixture of target nucleic acid molecules, said probe and said primer remains in contact with the well.

Methods and devices herein are suitable for detecting and/or amplifying a small number of copies, or even only a single copy, of target nucleic acid molecules, and can be used to detect and/or amplify a few or a single target molecule where a solution contains only a single type of target nucleic acid molecule or where a solution contains mixtures of multiple target molecules, where the separation and dilution of the sample solution allows detection of a few or of an individual target nucleic acid molecule.

Devices, assemblies and systems may comprise probes and/or primer molecules. Thus, for example, probes and/or primer molecules may be deposited in a reaction chamber, or in an assay chamber, or may be deposited as part of the methods disclosed herein. Systems comprising optical assemblies, thermal assemblies and reaction assemblies (having reaction chambers for amplification of target nucleic acid molecules) are provided in which used reaction assemblies may be replaced to provide reusable devices. Systems comprising analytical assemblies and detection assemblies are provided in which an assay cartridge having assay chambers may engage a thermal assembly for amplification of target nucleic acid molecules.

Methods and devices herein are suitable for detecting and/or amplifying a small number of copies, or even only a single copy, of one target nucleic acid molecule while also detecting and/or amplifying a small number of copies, or even only a single copy, of one or more other target nucleic acid molecules, so that multiple targets may be detected and/or amplified. Thus, methods and devices disclosed herein are suitable for detecting and/or amplifying targets disposed in a solution containing mixtures of multiple target molecules.

These novel methods and devices are suitable for use with such assays as the Taqman® assay and other fluorescence assays to detect amplification products derived from a single target molecule. Identification is enhanced by spatial isolation (from other targets) of target molecules of interest. The methods and devices provide highly accurate quantification of target copy molecules resulting from the amplification steps, allowing highly accurate counting and comparison between assays. Embodiments of the methods and devices provide multiple assays in a single step, or only a few steps, which may include a sample preparation step. The novel methods and devices disclosed herein may provide results that are similar to colony growing methods (which take days), but instead take only one or a few tens of minutes.

### BRIEF DESCRIPTION OF THE DRAWINGS

FIG. 1A illustrates a system for detecting and/or amplifying small numbers of target nucleic acid molecules.

FIG. 1B illustrates a plan view of a channel device of the system of FIG. 1A.

FIG. 1C illustrates a cross-sectional view of a channel device of the system of FIG. 1A taken along line 1C-1C.

FIG. 1D illustrates a cross-sectional view of a channel device of the system of FIG. 1A taken along line 1D-1D.

FIG. 1E illustrates a cross-sectional view of a channel device of the system of FIG. 1A taken along line 1E-1E.

FIG. 2 illustrates mixing of probes and primers drawn by capillary action into a section of the channel device of FIG. 1.

FIG. 3 illustrates dispensing target into a target well of the device of FIG. 1 for mixing with probes and primers.

FIG. 4 illustrates cleavage of 5′ nuclease probes with thermal cycling of the device of FIG. 1 by extending primers to generate fluorescence around targets that match the probes.

FIG. 5 illustrates the use of a camera or scanner to count the number of amplified spots produced on the channel device of FIG. 1.

FIG. 6 illustrates an embodiment of a device including valves to regulate the flow of probes, primers, or targets and to promote better mixing of target with the probes and primers.

FIG. 7 illustrates the device of FIG. 1 where the channels are segmented into small wells after target has been added.

FIG. 8 illustrates diffusion of signal from matched target and probe molecules, with the signal confined to the segmented wells during cycling.

FIG. 9 illustrates signal locations in a device of FIG. 7, the locations being formatted by the matrix of wells, aiding detection and counting of signal.

FIG. 10A illustrates a system for detecting and/or amplifying small numbers of target nucleic acid molecules.

FIG. 10B illustrates a perspective view of a barrier device of the system of FIG. 10A including barriers configured to compartmentalize probes and primers to a specific substantially two-dimensional area.

FIG. 10C illustrates a cross-sectional view of the barrier device of FIG. 10A taken along line 10C-10C of FIG. 10B.

FIG. 11 illustrates the uniform diffusion of probes and primers into a gel layer on a the floor of a well of the barrier device of FIGS. 10A-10C.

FIG. 12 illustrates the addition of target to the wells of the barrier device of FIGS. 10A-10C.

FIG. 13 illustrates the diffusion of target into the gel layer of the barrier device of FIGS. 10A-10C.

FIG. 14 illustrates target amplification and fluorescence in the barrier device of FIGS. 10A-10C after thermal cycling.

FIG. 15 illustrates detection and counting of amplified target molecules in a barrier device of FIGS. 10A-10C with a camera.

FIG. 16 illustrates targets labeled with different color fluorophores in a barrier device of FIGS. 10A-10C.

FIG. 17 illustrates detection and counting of targets labeled with different color fluorophores in a barrier device of FIGS. 10A-10C using a camera.

FIG. 18A illustrates a system for amplification and/or detection of target nucleic acid molecules having a deep well device.

FIG. 18B is a perspective view of a deep well device.

FIG. 19A illustrates amplification of target molecules matching the probe sequence in a deep well device of FIGS. 18A and 18B.

FIG. 19B is a cross-sectional view of the device of FIG. 19A taken along line 19B-19B.

FIG. 20 illustrates detection and counting of the fluorescent spheres created by the methods and devices of FIG. 18 using a camera.

FIG. 21A illustrates a system including a device having a post with hydrophilic wells surrounded by a hydrophobic surface.

FIG. 21B illustrates an end of a post of the device of the system of FIG. 21A.

FIG. 22 illustrates the post of the device of FIGS. 21A and 21B being dipped into a liquid mixture of target, probes, and primers.

FIG. 23 illustrates that when the post is extracted from the mixture of FIG. 22, the holes of a post of the device of FIGS. 21A and 21B are filled with mixture of target, probes, and primers, but the hydrophobic surface is not.

FIG. 24 illustrates the immersion of a post of the device of FIGS. 21A and 21B into oil, allowing thermal cycling of the mixture of target, probes, and primers without evaporation.

FIG. 25 illustrates amplification and fluorescence of probes matching the target in wells of a post of the device of FIGS. 21A and 21B.

FIG. 26 illustrates a method of imaging by a camera of a post of the device of FIGS. 21A and 21B following removal from oil in order to detect and count the fluorescence in holes.

FIG. 27 illustrates an alternative method of imaging of fluorescence in holes of a post of the device of FIGS. 21A and 21B with a camera collecting imaging information through a fiber bundle post.

FIG. 28 illustrates a device with hydrophilic holes in a plate with shallow walls that separate the plate into wells.

FIG. 29 illustrates addition of probes and primers to a well of the device of FIG. 28.

FIG. 30 illustrates probes and primers dried in a well of the device of FIG. 28.

FIG. 31 illustrates inversion of the plate of the device of FIG. 28.

FIG. 32 illustrates insertion of the plate of the device of FIG. 28 into a large well having target within the well.

FIG. 33 illustrates extraction of the plate of the device of FIG. 28 from the large well, collection of target, and mixture of the target with the probes and primers in the holes of the plate.

FIG. 34 illustrates the plate of the device of FIG. 28 inserted into oil such that the mixture of target, probe, and primer can be thermal cycled without evaporation.

FIG. 35 illustrates fluorescence of probes matching target molecules in the device of FIG. 28.

FIG. 36 illustrates imaging of the plate of the device of FIG. 28 with a camera for the detection and counting of targets.

FIG. 37 shows an example of a digital Taqman® reader having features of the invention.

FIG. 38 shows three assemblies of the digital Taqman® reader of FIG. 37 dis-assembled, enabling the removal of a used reaction assembly and insertion of a new reaction assembly.

FIG. 39A illustrates placement of a sample in a device for processing a sample for introduction into a reaction assembly having features of the embodiment of FIG. 38.

FIG. 39B illustrates mixing of a sample in a device for processing a sample for introduction into a reaction assembly having features of the embodiment of FIG. 38.

FIG. 39C illustrates flow of a mixed sample over capture beads in a device for processing a sample for introduction into a reaction assembly having features of the embodiment of FIG. 38.

FIG. 39D illustrates washing of capture beads in a device for processing a sample for introduction into a reaction assembly having features of the embodiment of FIG. 38.

FIG. 39E illustrates elution of sample DNA captured on capture beads in a device for processing a sample for introduction into a reaction assembly having features of the embodiment of FIG. 38.

FIG. 39F shows a side view of a reaction assembly having features of the embodiment of FIG. 38.

FIG. 39G shows a top view of a reaction assembly having features of the embodiment of FIG. 38.

FIG. 40A shows an example of an operation utilizing a reaction assembly having features of the embodiment of FIG. 38 in which sample material is added to the reaction assembly.

FIG. 40B shows an example of an operation utilizing a reaction assembly having features of the embodiment of FIG. 38 representing thermal cycling and unquenching operations with the reaction assembly.

FIG. 40C shows an example of an operation utilizing a reaction assembly having features of the embodiment of FIG. 38 in which reporters are illuminated to generate detectable fluorescence from the treated sample material in the reaction assembly.

FIG. 41A shows a side view of a further embodiment of a reaction assembly having features of the invention, the reaction assembly containing sample preparation chambers.

FIG. 41B shows a top view of a further embodiment of a reaction assembly having features of the invention, the reaction assembly containing sample preparation chambers.

FIG. 42A shows operations in a reaction assembly having features of the embodiment illustrated in FIGS. 41A and 41B, illustrating pipetting sample into the reaction assembly, mixing and lysing cells.

FIG. 42B shows operations in a disk cartridge having features of the embodiment illustrated in FIGS. 41A and 41B, illustrating electrophoresis of gDNA through a matrix and into buffer chambers.

FIG. 42C shows operations in a reaction assembly having features of the embodiment illustrated in FIGS. 41A and 41B, illustrating valve breakage and injection of gDNA into a reagent chamber by capillary action.

FIG. 42D shows operations in a reaction assembly having features of the embodiment illustrated in FIGS. 41A and 41B, illustrating thermal cycling and unquenching of reporters.

FIG. 42E shows operations in a reaction assembly having features of the embodiment illustrated in FIGS. 41A and 41B, illustrating illumination of reporters to generate detectable fluorescence from the treated sample material in the reaction assembly.

FIG. 43A illustrates application of a sample to an assay cartridge, and illustrates a thermal assembly, of an analytical assembly of a system having features of the invention.

FIG. 43B illustrates insertion of the assay cartridge into the thermal assembly of the analytical assembly of FIG. 43A.

FIG. 43C illustrates insertion of the assay cartridge of FIGS. 43A and 43B into a detection assembly of a system having features of the invention.

FIG. 44 is a perspective view of an assay cartridge having features of the invention.

FIG. 45A is a schematic illustration of a detection assembly of a system having features of the invention.

FIG. 45B is an enlarged view of the portion labeled 45B of the detection assembly of FIG. 45A having features of the invention.

FIG. 45C is a schematic illustration of an assay cartridge loaded within a detection assembly of a system having features of the invention.

FIG. 46A is a top view schematic illustration of a reaction module having features of the invention.

FIG. 46B is a cross-sectional schematic view of a reaction module having features of the invention taken along line 46B-46B of FIG. 46A.

FIG. 47A is a top view schematic illustration showing an initial step in the operation of a reaction module of FIGS. 46A and 46B.

FIG. 47B is a side view schematic illustration showing an intermediate step in the operation of a reaction module of FIGS. 46A and 46B.

FIG. 47C is a top view schematic illustration showing a later step in the operation of a reaction module of FIGS. 46A and 46B.

FIG. 48A is a schematic side view illustrating an initial step in the operation of a portion of an assay cartridge.

FIG. 48B is a schematic side view illustrating an intermediate step in the operation of a portion of an assay cartridge.

FIG. 48C is a schematic side view illustrating a later step in the operation of a portion of an assay cartridge.

### DETAILED DESCRIPTION

Methods and devices are presented in the Figures and described in the following. The Figures show configurations where nucleic acid target molecules are distributed at such a low concentration that a small number of copies, or even only a single copy, of a nucleic acid target molecule can be amplified and/or detected without mixing substantially with other targets. A single target may be identified, amplified, and/or quantified with methods and devices disclosed herein. Multiple targets disposed in a sample, or disposed in or on a device, may also be identified, amplified, and/or quantified with methods and devices disclosed herein. Nucleic acid dispersion can be accomplished by allowing solute diffusion through small bores or channels, by allowing solute diffusion through a thin gel layer, by having barrier segments keep nucleic acid targets separate for independent amplification and detection, or by other means. Multiple target nucleic acid molecules may be individually detected in pooled samples containing multiple targets (e.g., about 10 targets, or about 30 targets, or about 100 targets, or more).

Unless defined otherwise, technical and scientific terms used herein have the same meaning as is commonly understood by one of ordinary skill in the art to which this invention belongs. One skilled in the art will recognize many methods and materials similar or equivalent to those described herein, which could be used in the practice of the present teachings. Indeed, the present invention is in no way limited to the methods and materials described. For purposes of the present invention, the following terms are defined below.

As used herein, “target”, “target nucleic acid,” “target nucleic acid,” “target polynucleotide”, “target nucleic acid sequence,” “target sequence” and the like refer to a specific polynucleotide sequence that is the subject of hybridization with a complementary nucleic acid polymer (e.g., an oligomer). The nature of the target sequence is not limiting, and can be any nucleic acid polymer of any sequence, composed of, for example, DNA, RNA, substituted variants and analogs thereof, or combinations thereof. The target can be single-stranded or double-stranded. In primer extension processes, the target polynucleotide which forms a hybridization duplex with the primer may also be referred to as a “template.” A template serves as a pattern for the synthesis of a complementary polynucleotide. A target sequence may be derived from any living or once living organism, including but not limited to prokaryote, eukaryote, plant, animal, and virus, as well as non-natural, synthetic and/or recombinant target sequences.

As used herein, the term “probe” refers to a nucleic acid oligomer that is capable of forming a duplex structure by complementary base pairing with a sequence of a target polynucleotide, and further where the duplex so formed may be detected, visualized, measured and/or quantitated. In some embodiments, the probe is fixed to a solid support, such as in column, a chip or other array format.

As used herein, the term “primer” refers to a nucleic acid oligomer of defined sequence that hybridizes with a complementary portion of a target sequence and is capable of initiating the enzymatic polymerization of nucleotides (i.e., is capable of undergoing primer extension). A primer, by functional definition, is enzymatically extendable.

The term “primer extension” means the process of elongating an extendable primer that is annealed to a target in the 5′→3′ direction using a template-dependent polymerase. The extension reaction uses appropriate buffers, salts, pH, temperature, and nucleotide triphosphates, including analogs and derivatives thereof, and a template-dependent polymerase. Suitable conditions for primer extension reactions are well known in the art. The template-dependent polymerase incorporates nucleotides complementary to the template strand starting at the 3′-end of an annealed primer, to generate a complementary strand.

The terms “annealing” and “hybridization” are used interchangeably and mean the base-pairing interaction of one polynucleotide with another polynucleotide that results in formation of a duplex or other higher-ordered structure. The primary interaction is base specific, i.e., A/T and G/C, by Watson/Crick and Hoogsteen-type hydrogen bonding.

The term “sample” as used herein is used in its broadest sense. A “sample” is typically, but not exclusively, of biological origin, and can refer to any type of material obtained from animals or plants (e.g., any fluid or tissue), cultured cells or tissues, cultures of microorganisms (prokaryotic or eukaryotic), any fraction or products produced from a living (or once living) culture or cells, or synthetically produced or in vitro sample. A sample can be unpurified (e.g., crude or minimally processed) or can be purified. A purified sample can contain principally one component, e.g., total cellular RNA, total cellular mRNA, cDNA or cRNA. In some embodiments, a sample can comprise material from a non-living source, such as synthetically produced nucleic acid polymers (e.g., oligomers).

As used herein, the term “polymerase extension” refers to any template-dependent polymerization of a polynucleotide by any polymerase enzyme. It is not intended that the present invention be limited to the use of any particular polymerase. A polymerase can be an RNA-dependent DNA polymerase (i.e., reverse transcriptase, e.g., Moloney murine leukemia virus [MMLV] reverse transcriptase), DNA-dependent RNA polymerase (e.g., T7 polymerase, SP6 polymerase, T3 polymerase), or a DNA-dependent DNA polymerase (e.g., Taq DNA polymerase, Bst DNA polymerase, Klenow fragment, SEQUENASE™). A polymerase may or may not be thermostable, and may or may not have 3′→5′ exonuclease activity. Polymerase extension is not limited to polymerase activity that requires a primer to initiate polymerization. For example, T7 RNA polymerase does not require the presence of a primer for polymerase initiation and extension.

As used herein, the term “amplification” refers generally to any process that results in an increase in the amount of a molecule. As it applies to polynucleotides, amplification means the production of multiple copies of a polynucleotide, or a part thereof, from one or few copies or small amounts of starting material. For example, amplification of polynucleotides can encompass a variety of chemical and enzymatic processes. The generation of multiple DNA copies from one or a few copies of a template DNA molecule during a polymerase chain reaction (PCR) is a form of amplification. Amplification is not limited to the strict duplication of the starting molecule. For example, the generation of multiple RNA molecules from a single DNA molecule during the process of transcription (e.g., in vitro transcription) is a form of amplification.

Devices, systems and methods disclosed herein are useful for detecting, counting and/or amplifying a few copies or even a single copy of a target polynucleotide sequence. Such polynucleotide sequences may be detected, for example, using Taqman® procedures. Fluid samples containing nucleic acids having target sequences are applied to devices herein, where primers, probes, and other PCR reagents may be used to amplify target nucleic acid sequences present in the samples. For example, in some embodiments, nucleic acids within a sample may be separated by flow within or along a channel. Barriers may be put into place to prevent mixing after separation. Amplification of the separated nucleic acids by PCR results in separated or isolated populations of amplified nucleic acids, where each nucleic acid population is derived from a small number of, or even only a single, nucleic acid molecule. In other embodiments, nucleic acids within a sample are allowed to separate by diffusion into a gel, resulting in separated or isolated populations of nucleic acids derived from a small number of, or even only a single, nucleic acid molecule. The nucleic acid molecule may be a target nucleic acid molecule. The gel may be thick, or may be a thin gel layer, as discussed in greater detail beow. In some embodiments, nucleic acids within a sample are separated into a well or wells, resulting in separate populations of nucleic acids derived from a small number of, or even only a single, nucleic acid molecule.

Such populations of copies of target polynucleotides may be detected by optical or by other methods. Detection may be after amplification by, e.g., PCR. Polynucleotides comprising copies of a single or a small number of target polynucleotide(s) may be recovered from such regions for analysis, sequencing, or further amplification or processing. Devices, systems and methods herein offer the advantages of detection of a small number of copies, or even of a single copy of a target nucleic acid molecule. Thus, devices, systems and methods herein may be used to amplify and to detect less than about 10 target nucleic acid molecules, or less than about 5 target nucleic acid molecules, or a single target nucleic acid molecule. Devices, systems and methods herein may be used to amplify and to detect between about 5 and about 10 target nucleic acid molecules, or between about 3 and about 5 target nucleic acid molecules, or between about 1 and about 3 nucleic acid molecules, or a single target nucleic acid molecule.

Amplification of single nucleic acid molecules and of populations of nucleic acid molecules is typically performed by polymerase chain reaction (PCR) in which repetitive thermal cycles of heating and cooling of solutions containing nucleic acid molecules in the presence of a thermostable polymerase (e.g., Taq polymerase), primers, nucleotides, and other reagents results in the production of multiple copies of target nucleic acid molecules. For example, thermal cycles may comprise a denaturation portion, having a temperature typically between about 73° C. and about 99° C., or between about 85° C. and about 98° C., or between about 90° C. and about 97° C., or between about 93° C. and about 96° C.; an extension portion having a temperature typically between about 55° C. and about 72° C., or between about 60° C. and about 70° C., or between about 62° C. and about 68° C.; and an annealing portion having a temperature between about 22° C. and about 55° C., or between about 37° C. and about 55° C.

Methods for a wide variety of PCR applications are known in the art, and described in many sources, for example, Ausubel et al. (eds.), Current Protocols in Molecular Biology, Section 15, John Wiley & Sons, Inc., New York (1994); Mullis et al. (Methods in Enzymology 155:335-350 (1987); Martin et al., (Methods in Enzymology 305:466-476 (2000); U.S. Pat. Nos. 4,683,195, 4,683,202, 4,800,159 and 4,965,188.

Reverse transcriptase PCR (RT-PCR) is a PCR reaction that uses RNA template and a reverse transcriptase to first generate a DNA template molecule prior to the multiple cycles of DNA-dependent DNA polymerase primer elongation. Multiplex PCR refers to PCR reactions that produce more than one amplified product in a single reaction, typically by the inclusion of more than two primers in a single reaction. “Real-Time PCR” refers to PCR methods which allow monitoring of the progress of the reaction as the reaction proceeds. Such monitoring may occur between thermal cycles, or during a thermal cycle, and typically uses optical methods for detecting the presence of double-stranded nucleic acids. For example, see Klein, Trends in Molecular Medicine, 8(6):257-260 (2002). “Quantitative PCR” refers to PCR methods that provide an indication or measurement of the actual numbers of copies of nucleic acids produced by the amplification procedures, as opposed to relative numbers commonly obtained with other PCR methods. See, for example, Gilliland et al., PNAS 87:2725-2729 (1990) and methods discussed in Jung et al., Clin. Chem. Lab. Med. 38(9):833-839 (2000); Martin et al., Meth. Enzymol. 305:466-476 (2000); and Klein, supra.

In some embodiments, detection methods employ a labeled probe with the amplified DNA in a hybridization assay. For example, one method (termed “in situ hybridization”) uses a complementary single stranded DNA probe to which a label molecule has been attached. This probe is hybridized to the specific DNA sequences in the amplified sample, if there are any, and the excess probe and label is washed away. Then the locations of the remaining label molecules are rendered visible by treatment with developer reagents. For example, a label molecule on the amplified DNA may be biotin, and the binding molecule, coupled to the enzyme, avidin; or, the label molecule may be digoxigenin, and the binding molecule an anti-digoxigenin antibody. The labels on the label molecules may also be colored, fluorescent, or radioactive.

Another suitable PCR method is the “TAQMAN®” 5′ nuclease method. A “Taqman®” probe is a probe having a fluorescent indicator moiety attached internally or at one end (typically the 5′ end of the probe), and a quencher moiety attached internally or at an end (e.g., where a fluorescent indicator is attached at one end, a quencher moiety may be attached at the other end). When attached to the probe the proximity of the fluorescent indicator to the quencher prevents significant fluorescence from being emitted (due to fluorescence resonance energy transfer (FRET), or Forster-type energy transfer) or by non-resonance mechanisms. The probe is designed to be complementary to a sequence on a target polynucleotide, so that it anneals to a portion of a nucleic acid strand that is duplicated by PCR. As the primer is extended by DNA polymerase during PCR, the probe is cleaved so that the fluorescent moiety and the quencher moiety are no longer bound together by an intact probe. Thus, after the probe has been degraded by the action of DNA polymerase, the fluorescent moiety and the quencher moiety become separated, allowing emission of fluorescence. A threshold level of fluorescence may be determined or assigned as a measure of the progress of PCR amplification. Typically, a threshold level is defined at a level above which a logarithmic plot of fluorescence increases linearly with cycle number.

In some embodiments, 5′ nuclease assays and other assays are useful in “real-time” PCR techniques. Such techniques and methods allow the detection of amplified target nucleic acid molecules and the measurement of the progress of the PCR reaction. Such techniques typically do not require opening the reaction vessel in order to monitor PCR progress. By, for example, monitoring fluorescence emitted from the reactants, non-invasive and potentially continuous measurement is possible (see, e.g., Klein, Trends in Molecular Medicine, 8(6):257-260 June 2002, or Real-Time PCR—An Essential Guide, K. Edwards et al., Eds, Horizon Bioscience, Norfolk, UK (2004)). Exemplary methods for 5′ nuclease-mediated cleavage of Taqman®-type probes can be found, for example, in PCT Publication No. WO 96/15270 (Livak et al.), wherein fluorescence can be sampled during the denaturation step of each thermal cycle. Other real time PCR protocols are described for example in WO 96/34983 (Mayrand), WO 99/37670 (Coull et al.), WO 95/13399 (Tyagi et al.), and WO 01/94638 (Chen et al.). In such methods, a probe (containing a fluorescer moiety and a quencher moiety at opposite ends of the probe) is annealed to a target strand prior to the extension step of PCR (at an annealing temperature that is less than the primer extension temperature in the PCR cycle), and the resultant fluorescence can be measured as an indication of the amount of amplification at a particular thermal cycle. Typical “real-time PCR” indicators that are not sequence specific comprise, e.g., ethidium bromide, propidium iodide, SYBR™ Green I and II, PicoGreen™, and the Hoechst 33258 Dye. Ethidium bromide is a fluorescent compound that fluoresces while bound to double-stranded DNA. Other exemplary “real time” methods are described in, e.g., Holland, et al., PNAS 88:7276-7280 (1991), Higuchi et al. (Biotechnology 10:413-417 (1992), Higuchi et al. U.S. Pat. No. 6,171,785, Gelfand et al., U.S. Pat. No. 5,210,015, and Fisher, et al. (U.S. Pat. No. 5,491,063).

All patents and publications referred to herein, both supra and infra, are hereby incorporated herein by reference in their entireties.

Fisher et al. (U.S. Pat. No. 5,491,063) provides an assay that allows the simultaneous detection of the accumulation of amplified target and the sequence-specific detection of the target sequence. The method of Fisher et al. provides a reaction that results in the cleavage of single-stranded oligonucleotide probes labeled with a light-emitting label. The reaction is carried out in the presence of a DNA binding compound that interacts with the label to modify the light emission of the label. The method utilizes the change in light emission of the labeled probe that results from degradation of the probe to detect the presence of target molecules and of copies of target molecules. The methods are applicable in general to assays that utilize a reaction that results in cleavage of oligonucleotide probes, and in particular, to homogeneous amplification/detection assays where hybridized probe is cleaved concomitant with primer extension.

PCR can be used to amplify and/or detect a small number of target nucleic acid molecules. As used herein with reference to the novel systems, devices and methods, a small number of target nucleic acid molecules refers to less than about 10 target nucleic acid molecules, or to less than about 5 target nucleic acid molecules, or to a single target nucleic acid molecule. Thus, a small number of target nucleic acid molecules may be between about 5 and about 10 target nucleic acid molecules, or between about 3 and about 5 target nucleic acid molecules, or between about 1 and about 3 nucleic acid molecules, or may be a single target nucleic acid molecule.

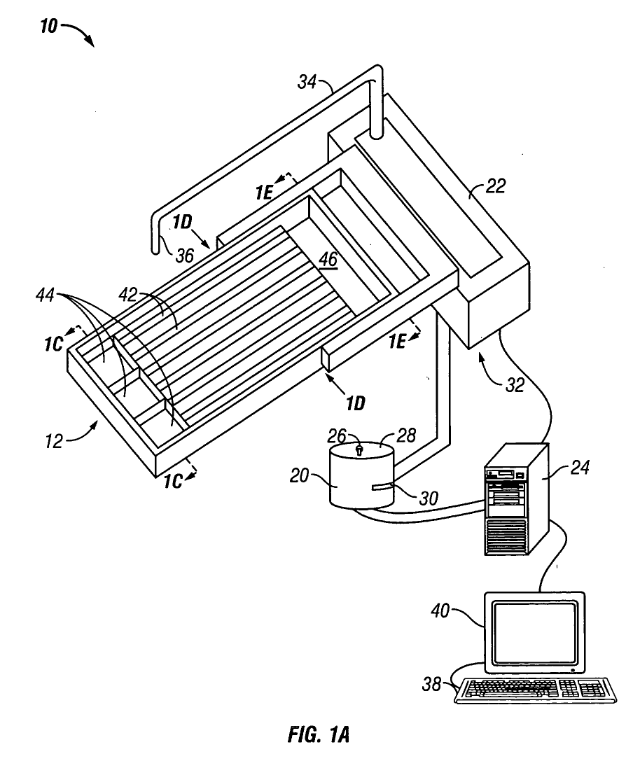

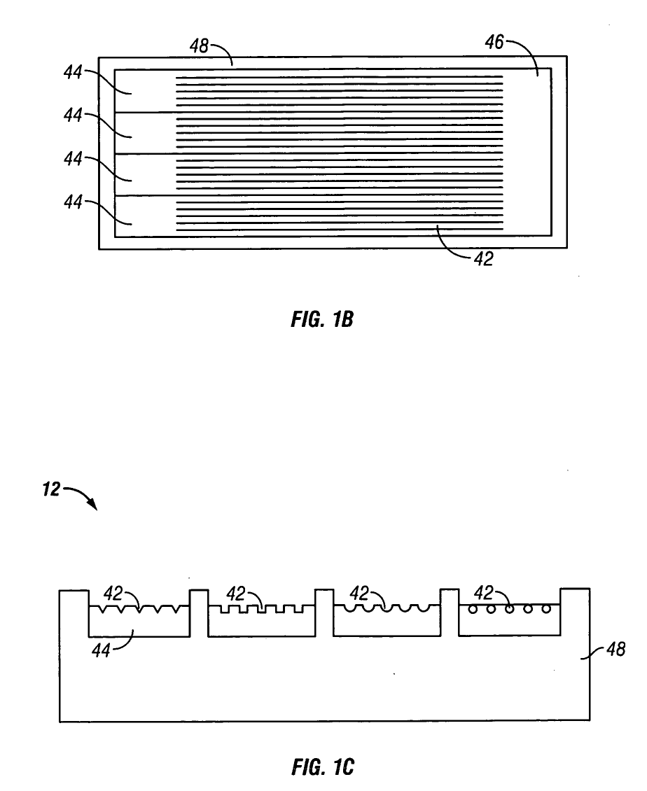

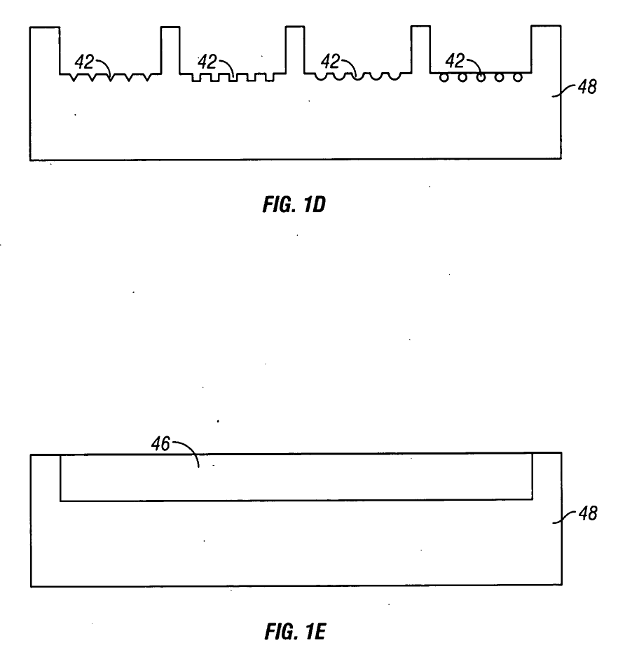

FIG. 1A illustrates a system 10, the system 10 being configured for amplifying and/or detecting a small number of target nucleic acid molecules. The system 10 shown in FIG. 1A comprises a channel device 12 that is configured to compartmentalize probes 14 and primers 16 (directed to a target nucleic acid molecule 18) to a specific area or location. Probes 14 may be Taqman® probes, for example. System 10 and device 12 are configured to perform PCR amplification of target nucleic acid molecules 18, being capable of functions and operations including thermal cycling (e.g., heating, cooling), mechanical transport or translation of system and device elements, solution handling, illumination, detection of fluorescence, data acquisition, data storage, and/or data output.

Several views of the channel device 12 are illustrated in FIGS. 1B-1E. Also shown in FIG. 1A are detector 20, mechanical apparatus 22, and controller 24. Detector 20 may comprise an optical detector, and may comprise an illumination source 26 and a lens 28 or other means for directing, focusing or collecting light or other radiation. Mechanical apparatus 22 comprises a mount 30 for holding a channel device 12. Mount 30 is operatively connected to a motor unit 32, which also has an arm 34 which is configured to carry and place dispenser 36 at a desired location with respect to channel device 12 for dispensing a liquid (e.g., a solution containing a probe 14, primer 16, target nucleic acid 18, or mixture of these). It will be understood that other mechanisms and means for delivering and dispensing liquids would also be suitable. Liquids are typically PCR buffers and other solutions useful for PCR techniques, and may contain probes 14, primers 16, target nucleic acid molecules 18, and other ingredients, or may be free of probes 14, primers 16, target nucleic acid molecules 18 (e.g., a wash solution). Solutions may comprise buffers, salts, and/or other constituents useful for performing PCR. Controller 24 may comprise a computer or other device having computational, data acquisition, data storage, and data analysis capability. Controller 24 also comprises input 38 (shown as a keyboard) and output 40 (shown as a computer monitor) devices or modules.

Channel device 12 has multiple channels 42 connecting with probe/primer wells 44 and with a target well 46. All probe/primer wells 44 connect with at least one channel 42. Some, but not necessarily all, channels 42 connect with a particular probe/primer well 44. All channels 42 connect with target well 46, so that a connection exists via a channel 42 between target well 46 and each probe/primer well 44.

As indicated in FIGS. 1C and 1D, channels 42 may have triangular, rounded, elliptical, square, rectangular or other cross-sectional shapes. Channels 42 may be open along a portion of their length, or may be enclosed. A channel 42 that is enclosed may be an enclosed tube with a circular or elliptical cross-section, or may have a triangular, square. Rectangular, or other cross-sectional shape. A device 12 may have only a single type of channel 42, or may have channels 42 having a variety of cross-sectional shapes. Similarly, wells 44 and 46 may have square cross-sections as shown, or may have triangular, rounded, elliptical, rectangular, or other cross-sectional shapes.

Channels 42 and wells 44 and 46 may be formed by cutting, etching, or otherwise removing material from a substrate 48, or by pressing or compressing a substrate 48 (which may first be heated or softened) to obtain the desired configuration. Channels 42 and wells 44 and 46 of a device 12 may also be formed at the same time as the substrate 48, by molding or casting a material to have such depressions. Alternatively, a channel 42 may be a tube or other structure having a bore. Thus, for example, a channel 42 may be formed by placing a tube or other material having a bore onto or into a substrate 48, or combining such material to form a substrate 48 with a bore or bores. Such tubes may be capillary tubes. Alternatively, channels 42 and wells 44 and 46 may be formed on top of a substrate 48, or by placing a wall or walls onto a substrate 48, or may be formed in part by cutting into a substrate 48 and in part by building up at least a portion of a substrate 48. A substrate 48 may be translucent (i.e., allowing the passage of electromagnetic radiation) or may be transparent (i.e., allowing the passage of electromagnetic radiation effective to allow localization of a source of electromagnetic radiation within or on the other side of the substrate 48, such as allowing the formation of an image from electromagnetic radiation passing through the material). Alternatively (e.g., where a detector 20 is disposed opposite a channel 42 or an uncovered face of a well 44 or 46), the substrate 48 need not be transparent, while the channel 42 may be transparent to at least a portion of the electromagnetic spectrum or may be open on a portion disposed opposite a detector 20.

The channels 42 may have small dimensions so that fluid within the channels flows slowly, limiting the rate of dispersion of target nucleic acids present in liquids within the channels. However, although slow, such fluid flow is enhanced by capillary action due to the size and configuration of the channels. In some embodiments, an elongated channel has a width of less than about 10 mm, or less than about 5 mm, or less than about 2 mm, or less than about 1 mm, or less than about 0.5 mm, or less than about 0.2 mm. Thus, in some embodiments, an elongated channel may have a width of between about 5 mm and about 10 mm; or between about 2 mm and about 5 mm; or between 1 mm and about 2 mm; or between about 0.5 mm and about 1 mm, or between about 0.2 mm and about 0.5 mm, or more than 0 mm and less than about 0.2 mm. In some embodiments, an elongated channel has a depth of less than about 5 mm, or less than about 2 mm, or less than about 1 mm, or less than about 0.5 mm. Thus, in some embodiments, an elongated channel may have a depth of between about 2 mm and about 5 mm; or between about 2 mm and about 2 mm; or between 0.5 mm and about 1 mm; or less than about 0.5 mm.

In some embodiments, an elongated channel may have a width of about 0.3 mm and a depth of about 0.15 mm. In some embodiments, a device has multiple elongated channels, each having a width of about 0.3 mm and depths of about 0.15 mm. Channels 42 of channel device 12 may be about 0.3 mm wide by about 0.15 mm deep. Probe/primer wells 44 may be about 2 mm by about 2 mm; and a target well 46 may be about 2 mm by about 9 mm. In some embodiments, a channel 42 may have a length of up to about 30 cm, or may have a length of up to about 20 cm, or may have a length of up to about 10 cm, or may have a length of up to about 5 cm, or may have length of less than about 5 cm. Thus, a channel 42 may have a length of between about 20 cm and about 30 cm; or may have a length of between about 10 cm and about 20 cm; or may have a length of between about 5 cm and about 10 cm; or may have a length of less than about 5 cm.

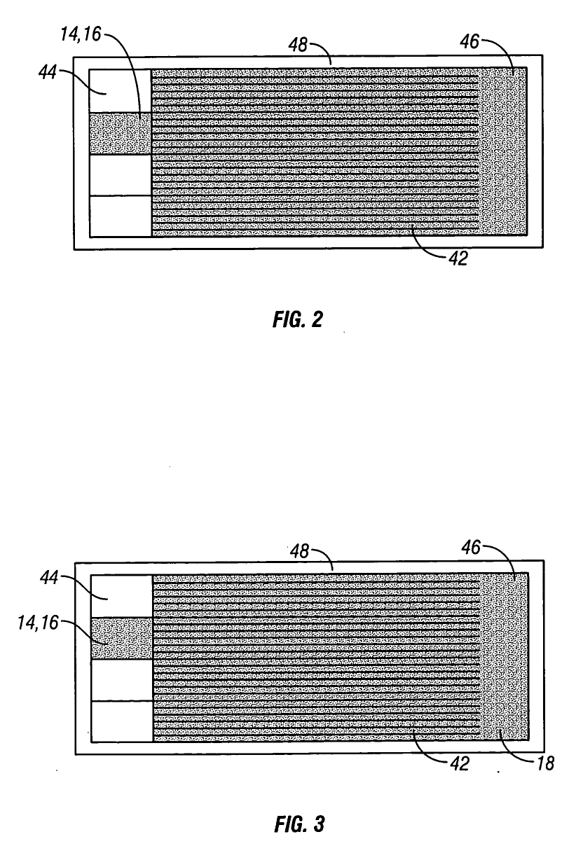

FIG. 2 illustrates a use of a channel device 12. A probe/primer well 44 is shown after a mixture of probes 14 and primers 16 has been dispensed into it. Such a mixture is typically provided as a solution that may also comprise other elements, such as salts, buffers, and/or other constituents useful for PCR. Capillary action is effective to draw the mixture from probe/primer well 44 into a section 50 of channels 42. Alternatively, the mixture may be drawn into a section 50 of channels 42 by suction or forced into a section 50 of channels 42 by pressure. As illustrated in FIG. 2, a section 50 may be a group of channels 42 that all connect with the same probe/primer well 44. The probes 14 and primers 16 are dried inside the channels 42 of section 50. Drying may be accomplished, for example, by application of heat, or by illumination by a heating lamp, or at room temperature, in air, or under an inert gas such as nitrogen or argon, optionally under reduced pressure, or by other suitable method or combination of methods. Probes 14 and primers 16 optionally may be dried within probe/primer well 44 as well as in channels 42. Multiple sections may be prepared. Each section may be prepared in a different manner, so that different and potentially multiple sets of probes and primers, i.e. different assays, or each section may be prepared in the same manner as other sections.

As illustrated schematically in FIG. 3, a solution containing target nucleic acid molecules 18 may be placed into the target well 46 (e.g., by dispenser 36). Target nucleic acid molecules 18 are drawn by capillary action into all the channels 42 of the device 12, mixing with the dried probes 14 and primers 16 in the channels 42 of section 50. Alternatively, flow of solution containing target nucleic acid molecules 18 may be effected or aided by suction, pressure or by other means instead of or in addition to capillary action. Such diffusion into the channels 42 may also serve to separate and dilute the target nucleic acid molecules 18 so that there may be at most one, or at most a small number of target nucleic acid molecules 18 at a given region 52 within a channel 42. Many locations within a channel 42 will have no target nucleic acid molecules 18, so that target nucleic acid molecules 18 will be, in general, separated from one another following dispersion within a channel 42. With sufficient dilution of target nucleic acid molecules 18 in target well 46 and with sufficient separation of such target nucleic acid molecules 18 as they move within channels 42 by capillary action or otherwise, conditions may be provided in which only a single copy of a target nucleic acid molecule 18 will be found within a region 52 in a channel 42. It will be understood that solutions may be applied in any order, and that, for example, target nucleic acid molecules 18 may be applied before application of probes 14 and primers 16.

The presence of probes 14, primers 16, and target nucleic acid molecules 18 within channels 42 in a solution suitable for PCR provides conditions in which amplification of target nucleic acid molecules 18 may be performed. Such a solution may comprise buffers, salts, and/or other constituents useful for performing PCR. A device 12 and a system 10 are each configured for thermal cycling suitable for PCR amplification of target nucleic acid molecules 18. The number of copies of target nucleic acid molecules 18 increases with each thermal cycle. The rate of this increase may decline if the availability of nucleotides, primers or probes becomes limiting. Since within the channels 42 target nucleic acid molecules 18 are typically found in isolated and separate regions 52, initially (i.e., before thermal cycling) with one, or with a small number of copies of target nucleic acid molecules 18 in each region 52, with succeeding PCR thermal cycles multiple copies of target nucleic acid molecules 18 increase within such separate regions 18. Thus, within each separate region 52, all copies of target nucleic acid molecules 18 will be derived from a single original copy of a target nucleic acid molecule 18, or from a small number of original copies of target nucleic acid molecules 18.

Amplification of copies of target nucleic acid molecules is often plotted on an “amplification plot” in which the fluorescence signal detected from amplified nucleic acid molecules is plotted against the cycle number. The thermal cycle at which the fluorescence signal passes a set threshold may be termed the “threshold cycle” and is often denoted “C_{T}.” A useful threshold is where the fluorescence signal becomes detectable (indicating that amplification of target nucleic acid molecules is detectable). Fluorescence (or other signal) from copies of target nucleic acid molecules 18 may be measured within each separate region 52 to measure the amount of amplification and the progress of the amplification of target molecules. An amplification plot of the signal may be made, and the signal compared to a C_{T} effective that the amplification of target nucleic acid molecules may be quantified. Other methods of detecting and analyzing the amplification of target molecules may also be employed.

Copies of target nucleic acid molecules 18 and indicator (e.g., dyes) will tend to diffuse away over time from their initial location within a region 52. With multiple cycles, such diffusion might lead to overlap between different regions 52 and to mixing of copies of target nucleic acid molecules 18 that are derived from different original copies of the target nucleic acid molecule 18. It is desirable to reduce such diffusion in order to reduce the overlap or mixing of copies of target nucleic acid molecules 18 within a channel 42. In order to reduce the diffusion distance, thermal cycles may be fast, or few, or fast and few. Thus, the duration of the heating phase of a thermal cycle may be short, or the duration of the cooling phase of a thermal cycle may be short, or both. The numbers of thermal cycles may be kept to a minimum in order to reduce the dispersal of copies of target nucleic acid molecules 18 and of indicator dyes. Use of a viscous solution and/or matrix may also be effective to reduce the diffusion distance, either alone or in combination with fast and/or few thermal cycles. A viscous buffer has a viscosity greater than that of water, that is, greater than about 1 centipoise (g/cm-s) at room temperature. A viscous buffer thus has a viscosity of greater than 1 centipoise, or greater than about 2 centipoise, or greater than about 10 centipoise, and may be greater than about 100 centipoise, greater than about 500 centipoise, or greater than about 5000 centipoise at room temperature.

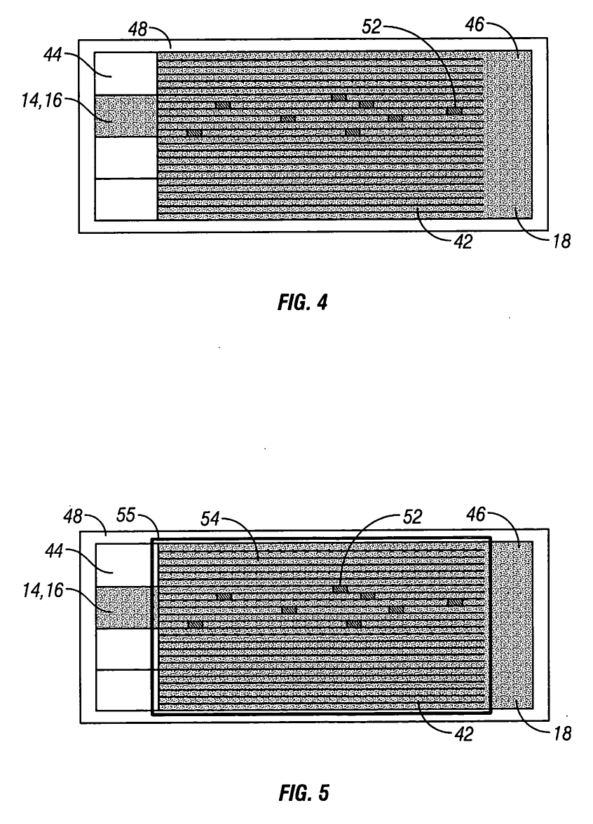

FIG. 4 illustrates a device 12 after it has been thermal cycled such that Taqman® probes 14 are cleaved by extending primers 16 to generate fluorescence near target nucleic acid molecules 18 that match the probes 14. Such fluorescence indicates amplified target matches in regions 52, illustrated in FIG. 4 by the dark rectangles at locations within channels 42. Thus, in a device 12 used according to methods disclosed herein, separate regions 52 within channels 42 will contain amplified target matches. Such regions 52 containing multiple copies of target nucleic acid molecules 18 are produced by amplification of a single copy of a target nucleic acid molecule 18, or by amplification of as few as a small number of target nucleic acid molecules 18 that were initially present in a region 52 before initiation of PCR amplification.

Real-time PCR techniques may be used to allow the detection of amplified target nucleic acid molecules 18. For example, amplification of target nucleic acid molecules 18 may cause degradation of probes 14 and thereby release of indicator dyes. Release of fluorescent indicator dyes is detectable by a detector 20 of a system 58 after amplifying by PCR. The indicator dye may be from Taqman® probes for use in a PCR system to indicate amplification of target molecules 18 complementary to probes 14.

As illustrated in FIG. 5, shown in a plan view of a portion of a system 10 which may be imaged or scanned by a detector 20 such as a camera, confocal microscope, or scanner is used to sense the presence or absence of fluorescence from a portion 54 of the device 12. The outer limits of a portion 54 of a system 10 that is imaged or scanned is indicated by dark outline 55 in FIG. 5. At least a portion of a substrate 48 and channels 42 are transparent to at least a portion of the electromagnetic spectrum. Substrate 48 is typically transparent to light, or to other forms of radiation useful for detecting indicators of amplification of target nucleic acid molecules 18. Channels 42 are also transparent or otherwise suitable for detection of amplification of target nucleic acid molecules 18. Such radiation suitable for detecting indicators of amplification of target nucleic acid molecules 18 may be optical radiation such as visible light, infrared light, or ultraviolet light, or electromagnetic radiation of other wavelengths. Alternatively, substrate 48 and channels 42 need not be transparent in some embodiments where imaging is performed by light or other radiation collected by imaging devices situated above a device 12 (e.g., where a detector 20 is located on the same side of the device 12 as arm 34 and dispenser 36 as illustrated in FIG. 1A).

A detector 20 may comprise a camera. A camera image of the entire portion 54 may be used in order to count the number of amplified spots, i.e. the number of regions 52 containing amplified target nucleic acid molecules 18. The camera image may be taken as soon as possible after the last thermal cycle in order to limit diffusion distance. Multiple assays may be performed and detected in a portion 54 using different probe/primer sets including different color fluorescent molecules for each probe type. For example, for each color, a separate picture may be obtained with an appropriate color filter for detection by an optical detection device, or separate colors may be detected simultaneously with different detection devices sensitive to different optical wavelengths, or a single detector sensitive to multiple wavelengths. Suitable optical detection devices comprise photomultiplier tubes, charge-coupled detector devices (CCDs), video cameras, and other optical devices. A detector 20 may comprise a microscope (e.g., a confocal microscope) and will typically comprise a lens, mirror or other optical collector, an optical detector, and associated power, controlling elements, input and output elements, and may also comprise a filter, a diaphragm or shutter, scanner, and other elements as well. A detector may comprise fiber optic components.

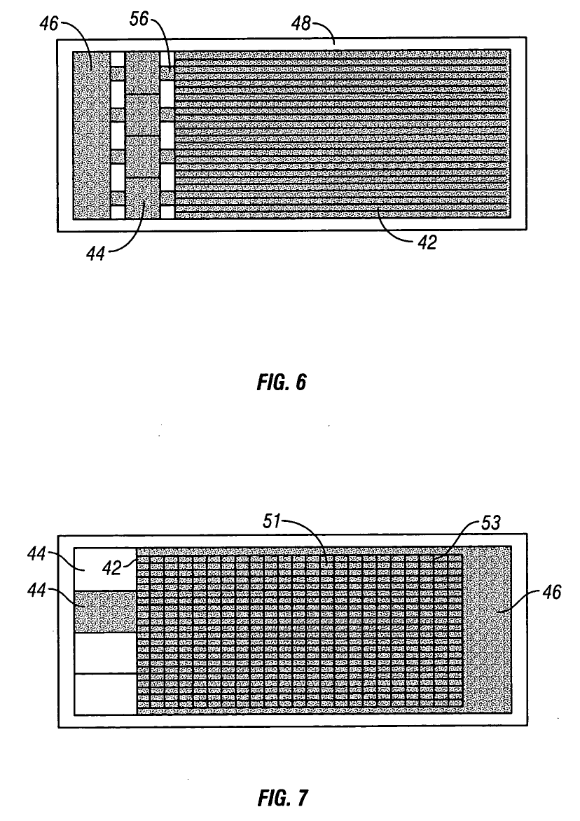

FIG. 6 illustrates a device 12 having valves 56 situated between probe/primer wells 44 and channels 42. Such valves may be used to regulate the flow of solutions and of probes 14 and primers 16, and to promote better mixing of target 18 with the probes 14 and primers 16. Opening of valves 56 may be triggered or controlled by, for example, centrifugal force, by heat, by electrical, by magnetic, by fluidic, by mechanical, or by other means or a combination of these or other means. For example, valves 56 may be polyethylene glycol (e.g., of a molecular weight selected to have the desired melting temperature and degradation characteristics), wax, polymer, or other material which may be removed or altered to allow passage of material by e.g., heat, or light, electrical power, or other controlling signal.

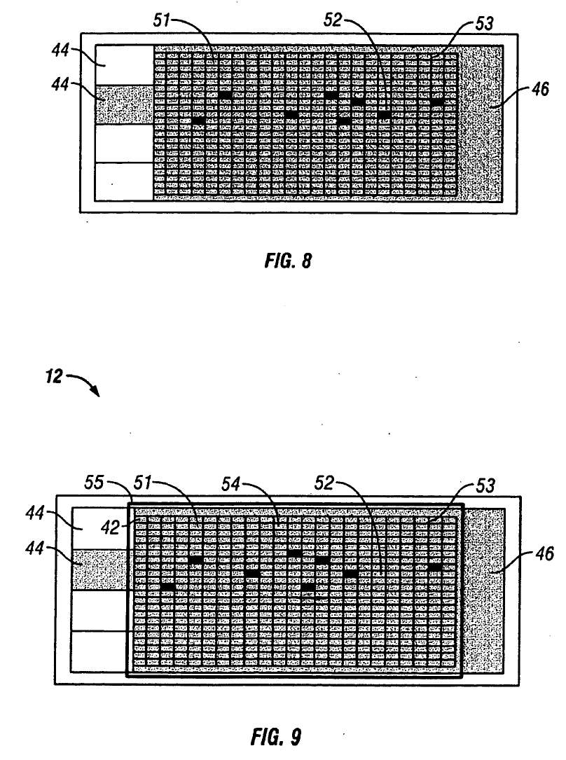

In some embodiments of a device 12 of a system 10, channels 42 may be segmented into small wells 51 after target molecules 18 have been added, as illustrated in FIG. 7. The segmenting may be accomplished by heat, mechanical means, electrical means, fluidic means, magnetic means, or by other or a combination of means. Dividers, such as barriers 53 illustrated in FIG. 8, may be situated in place after addition of target nucleic acid molecules 18; or after added target nucleic acid molecules 18 have traveled along channels 42; or at other times after addition of target nucleic acid molecules 18. Placement, activation or positioning of barriers 53 may be by means of centrifugal force, by heat, by electrical, by magnetic, by fluidic, by mechanical, or by other means or a combination of these or other means.

For example, barriers 53 may be used to segment channels 42 into small wells 51 so that regions of amplified target matches 52 remain separate and do not mix together. This is illustrated in FIG. 8, showing that diffusion of signal indicating amplification of target molecules 18 is confined to the segmented wells 51 during PCR cycling. The dark rectangular regions indicate regions 52 of amplified target matches, kept separate within small wells 51 by barriers 53. In some embodiments, small wells 51 are local depressions within a channel 42. FIG. 9 illustrates the detection and counting of amplified numbers of copies of target molecules 18 formatted by the matrix of wells. A detector 20 such as a camera is used to detect fluorescence and may be used to count regions 52 of amplified target matches. As indicated in FIG. 9 showing a camera image, a camera image of the entire portion 54 within outer limits 55 may be used in order to count the number of regions 52 defined by wells 51 containing amplified target nucleic acid molecules 18. Although the camera image may be taken as soon as possible after the last thermal cycle, barriers 53 are effective to limit diffusion distance and to prevent or inhibit mixing and dilution of the fluorescence signal so that an image taken later may also be useful.

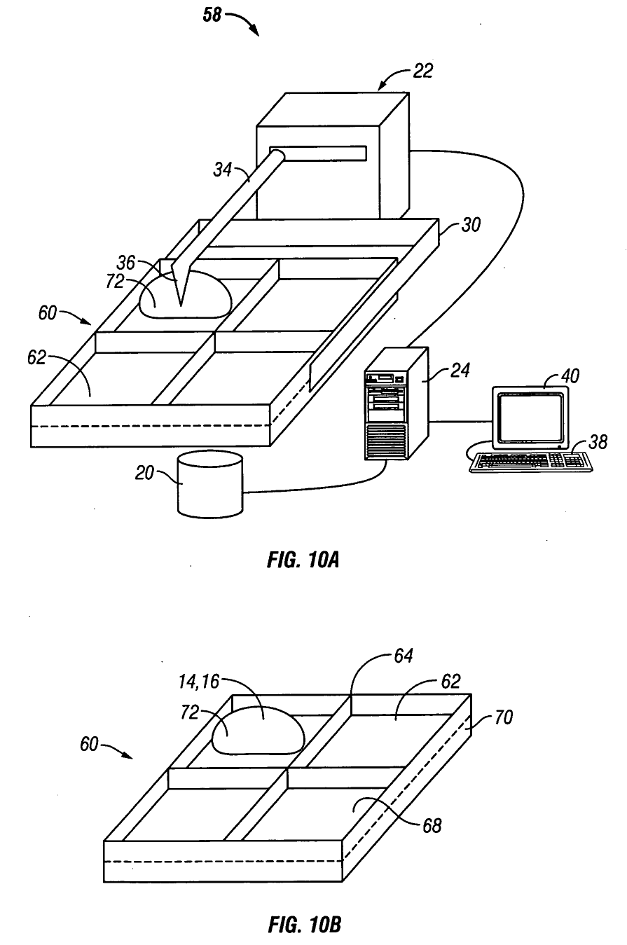

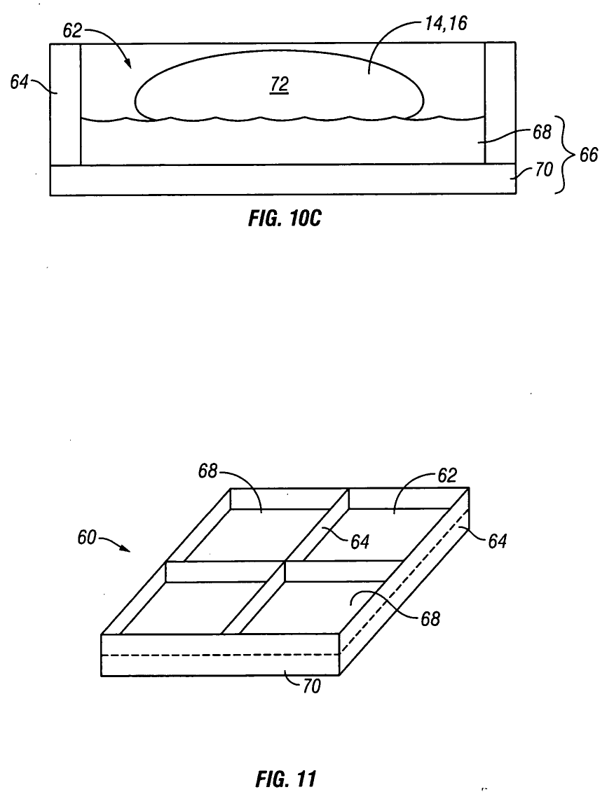

FIGS. 10A-10C illustrate a system 58 for amplifying and/or detecting small numbers of target nucleic acid molecules having a barrier device 60. Barrier device 60 has multiple wells 62 separated by barriers 64 and having a bottom 66 at least a portion of which is made of, or covered with, a bottom layer 68 configured to accept probes 14, primers 16 and target nucleic acid molecules 18 and to modulate the diffusion of copies of target nucleic acid molecules 18. A bottom layer 68 forms a base for a well 62. A bottom 66 may be, for example, a plate on which a bottom layer 68 rests or is supported. For example, bottom 66 may comprise a bottom layer 68 that is a thin gel layer (e.g., a thin acrylamide gel layer) on top of a substrate 70. A droplet 72 of solution containing probes 14 and primers 16 is shown resting on bottom layer 66. It will be understood that such a solution may also comprise other elements, such as salts, buffers, and other constituents useful for PCR. Barriers 64 can compartmentalize probes 14 and primers 16 to within a specific area (e.g., a well 62) that is substantially two-dimensional (i.e., has only a small depth as compared to dimensions of the length or width of the well 62). A pre-deposited, thin-layer of gel 68 is effective to slow diffusion of probes 14 during PCR cycling. Different probes 14 and primers 16 may be deposited into different wells 62 of a device 60 for simultaneous detection and/or amplification of different target molecules 18 in different wells 62.

After deposition of a droplet 72 onto gel layer 68, probes 14 and primers 16 diffuse into the gel layer 68 on the bottom 66 of a well 62 of a barrier device 60, as illustrated in FIG. 11. The probes 14 and primers 16 will typically diffuse uniformly into a gel layer 68, with uniformity of diffusion enhanced where the gel layer 68 is a thin gel layer. For example, a thin gel layer may be a gel layer having a thickness of less than about 0.5 mm, or less than about 0.1 mm, or less than about 0.05 mm. For example, in some embodiments of a system 58 and a barrier device 60, a suitable gel layer 68 is a thin acrylamide layer about 0.04 mm thick. The thin gel layer 68 is configured to reduce diffusion within it; vertical diffusion is limited physically by the thinness of the layer; lateral diffusion is also limited by the small vertical dimensions of the layer, and by the material of which it is made. For example, a thin gel layer 68 may be an acrylamide gel layer, or may be made from other gelatin, or other materials such as agar, agarose, acrylamide, Sepharose®, Sephadex®, Sephacryl®, casein, unfixed gels and cross-linked gels. A gel layer 68 may be made from a mixture of any such materials.

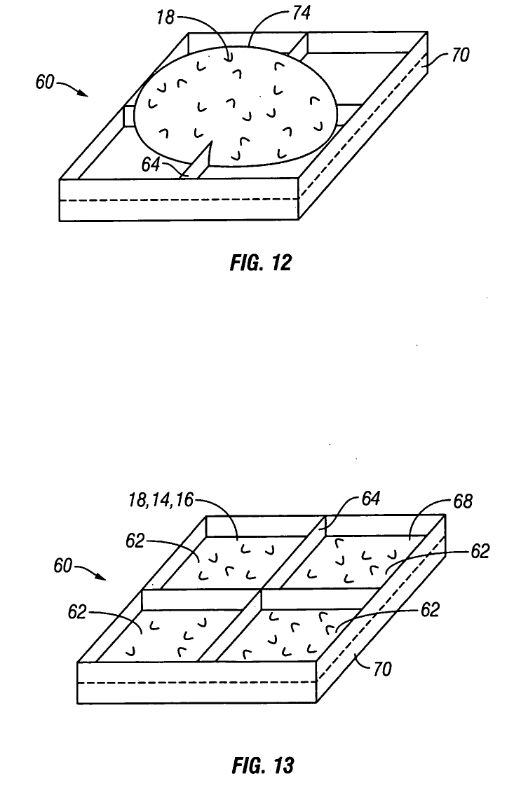

Following deposition of a droplet 72 (containing probes 14 and primers 16) into a well 62 and diffusion of probes 14 and primers 16 into a gel layer 68 on a bottom 66 of a well 62, a droplet 74 (containing target nucleic acid molecules 18) may be placed on a barrier device 60, as illustrated in FIG. 12. After addition of the target molecules 18 to wells 62, target molecules 18 may be allowed to diffuse into the gel layer 68 on the bottoms 66 of wells 62, as illustrated in FIG. 13. Target molecules 18 may be dried on the bottoms 66 of wells 62 and in the gel layer 68. Thus, gel layers 68 of wells 62 contain probes 14, primers 16 and the target molecules 18 as these materials diffuse and/or dry into the gel layers 68. Barriers 64 prevent the spread of probes 14, primers 16 or target molecules 18 from one well 62 to another. As illustrated in FIG. 13, following deposition onto, and diffusion into, gel layer 68, target molecules 18 are sparsely distributed within gel layers 68 of wells 62, separated from one another by regions having no target molecules 18.

Thermal cycling applied to a barrier device 60 having probes 14, primers 16 and target molecules 18 diffused into thin gel layers 68 of wells 62 is effective to amplify target molecules by PCR. As indicated in the Figures, the target nucleic acid molecules 18 are well separated, so that a single target nucleic acid molecule 18, or a small number of target nucleic acid molecules 18, are found at any one site in a well 62. The number of copies of target nucleic acid molecules 18 increases with each cycle (at least in the absence of depletion of primers 14, probes 16, or other necessary component of the PCR reaction), so that copies of target molecules 18 tend to diffuse away from the initial site where the initial target nucleic acid molecule 18. The thin gel layer 68 is configured to reduce the spread of copies of the target nucleic acid molecule 18 and of indicator dyes away from any one of the sites of initiation of the PCR amplification. Thus, regions 76 of amplified copies of target nucleic acid molecules 18 derived from a single, or from as few as a small number of, target nucleic acid molecules 18 arise and dot the bottom 66 of the wells 62.

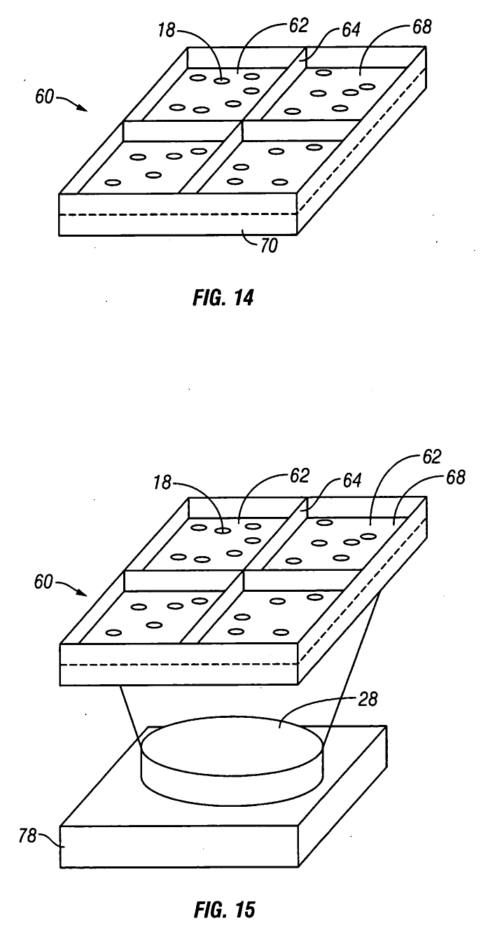

FIG. 14 shows such regions 76 of amplified target nucleic acid molecules 18 after thermal cycling. Release of fluorescent indicator dyes from probes 14 degraded by amplification of target nucleic acid molecules 18 by elongation of primers 16 is detectable by a detector 20 of a system 58 after amplifying by PCR.

In some embodiments, at least a portion of each of bottom 66, thin gel layer 68, and substrate 70 is transparent or translucent to at least a portion of the electromagnetic spectrum. Bottom 66, thin gel layer 68, and substrate 70 are typically transparent to light, such as visible light, infrared light, ultraviolet light, or electromagnetic radiation of other wavelengths suitable for detection of amplification of target nucleic acid molecules 18. Alternatively, bottom 66 and substrate 70 need not be transparent, in some embodiments where imaging is performed only through the thin gel layer 68 (which must then be transparent or translucent), as by imaging taken from above the thin gel layer 68.

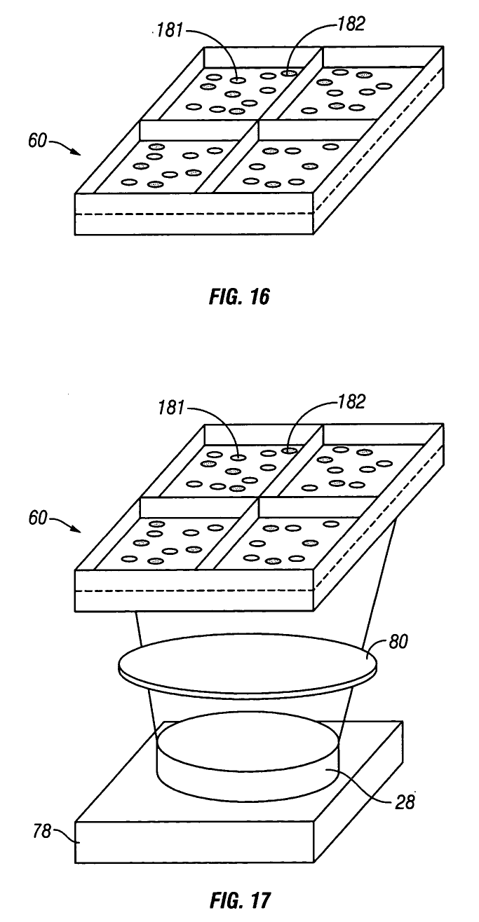

FIG. 15 illustrates detection and counting of target regions 76 with a detector that comprises a camera 78. Multiple target nucleic acid molecules 181 and 182 may be amplified and/or detected simultaneously, within a single well 62 or within different wells 62, with differently colored indicator dyes. For example, different probes 14 and primers 16 having different fluorophores attached to different probes 14 are useful to indicate which target nucleic acid molecule 18 complementary to a probe 14 had been amplified. FIG. 16 illustrates target regions 76 with different color fluorophores indicating amplification of two different target nucleic acid molecules 181 and 182. Such different fluorophores may be detected by a camera 78 by use of a color filter 80 as indicated in FIG. 17, showing detection and counting of targets 181 and 182 in target regions 76 labeled with different color fluorophores. A different color filter 80 may be used for each color fluorophore. Alternatively, a camera 78 may itself be able to detect different fluorophores without use of a color filter 80 or of multiple color filters 80.

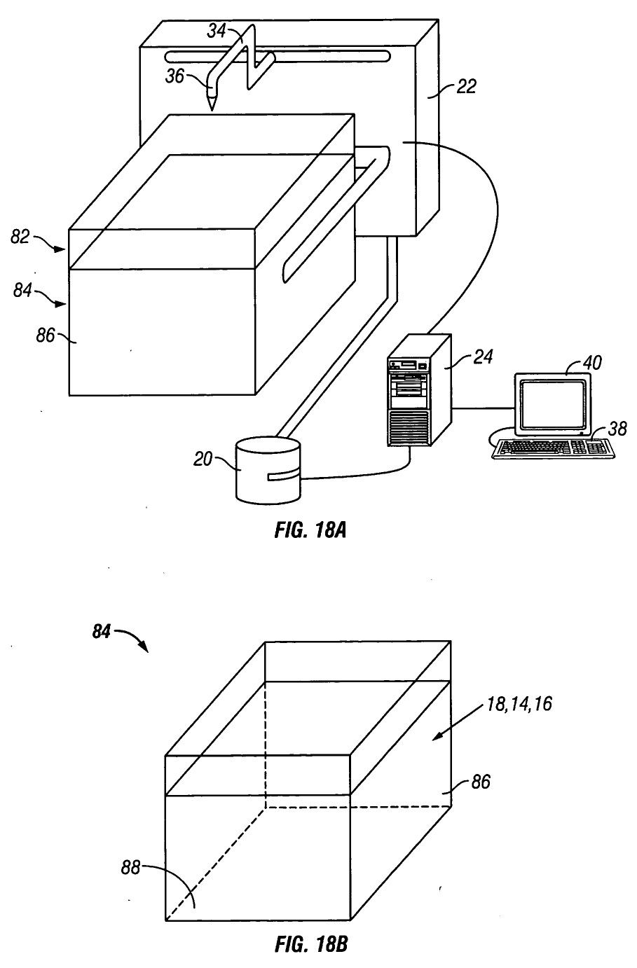

A system 82 having a device 84 with a deep well 86 containing a thick gel layer 88 is illustrated in FIG. 18A. A deep well 86 has a depth that is comparable in dimension to the length and width of the deep well 86. A perspective view of a device 84 is shown in FIG. 18B. Solutions suitable for use with PCR containing probes 14, primers 16 and target nucleic acid molecules 18 may be deposited onto a thick gel layer 88, as by a dispenser 36 shown in FIG. 18A. Probes 14 may be Taqman® probes. Probes 14, primers 16 and target nucleic acid molecules 18 may then diffuse into thick gel layer 88, where target nucleic acid molecules 18 may become widely separated. Such separation will disperse target nucleic acid molecules 18 into separate regions 90 spaced apart from each other.

Thus, a system 82 and device 84 are suitable for mixing target molecules 18, probes 14, and primers 16 within the volume of a well 86. A device 84 may be subjected to thermal cycling to amplify target nucleic acid molecules 18 within a deep well 86 by PCR. Amplification of target nucleic acid molecules 18 (e.g., by PCR using Taqman® probes) will produce large numbers of copies of the individual target nucleic acid molecules 18 within, and release indicator dyes into, a localized region 90 around the initial locations of the target nucleic acid molecules 18. The thick gel layer 88 serves to reduce the spatial dispersion of copies of target molecules 18 and indicator dye released during the copying of target molecules 18, thereby limiting the size and spread of small regions 90. Amplification of target molecules 18 can then readily be detected in small regions 90 within a deep well 86. Regions 90 containing copies of target nucleic acid molecules 18 produced by PCR amplification are detectable by fluorophores released from probes 14 as primers 16 are extended during PCR cycles. PCR thermal cycling creates roughly spherical regions 90 of fluorescence within the volume of a well 86.

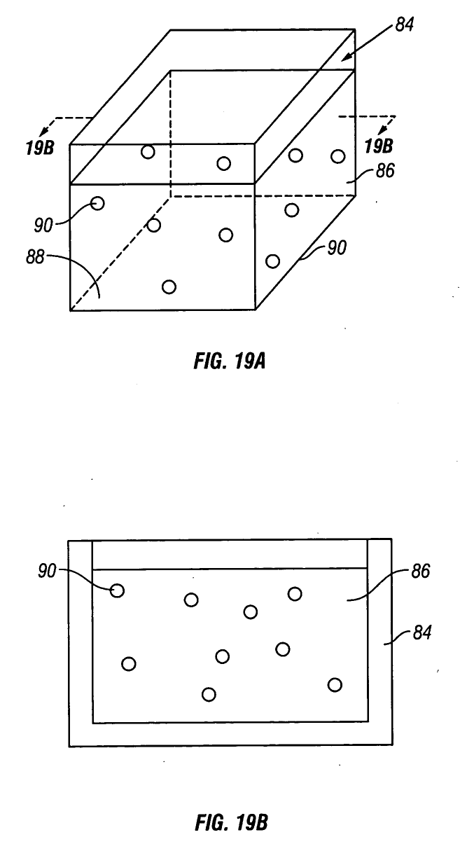

Such small regions 90 are illustrated in FIGS. 19A and 19B, with FIG. 19A showing a perspective view of a well 86 containing multiple roughly spherical small regions 90, and FIG. 19B showing a cross-sectional view through the thick gel layer 88 within a well 86 and through several small regions 90. The apparent diameter of the small regions 90 in a cross-sectional view varies depending on the position of the cross-section with respect to the center of a small region 90.

Roughly spherical regions 90 produced by amplification of target nucleic acid molecules 18 within the thick gel layer 88 in a deep well 86 of a device 84 may be detected and counted by optical means. In some embodiments, portions of a device 84, a deep well 86, and the thick gel layer 88 are transparent or translucent to at least a portion of the electromagnetic spectrum. Alternatively (e.g., where a detector 20 is disposed opposite an uncovered face of a gel layer 88), only the thick gel layer 88 is transparent or translucent to at least a portion of the electromagnetic spectrum. For example, as illustrated in FIG. 20, regions 90 may be detected and/or counted by a detector 20 by moving the image plane 92 of a camera 94 through the volume of the thick gel layer 88. Alternatively, along with or instead of a camera, a microscope, such as a confocal microscope, may be used to detect and or count regions 90. In some embodiments, vertical or horizontal cross-sections of a thick gel layer 88 may be imaged by a camera, microscope (e.g., confocal microscope), or other means to detect and quantify the presence and amount of amplification of target nucleic acid molecules 18.

A deep well 86 of a deep well device 84 is preferably deeper than a well 62, and a thick gel layer 88 is preferably thicker than a thin gel layer 68 of a device 60. Such a thick gel layer 82 may be in a device 84 having a plurality of wells 86 or may be in a device 84 having only a single well 86. Use of such an embodiment is similar to the use of a device 60, except that vertical diffusion of target nucleic acid molecules 18 and of indicator dyes is not substantially limited by the physical dimensions of the thick gel layer 82 or of the well 84.

For example, a thick gel layer 88 contained within a deep well 86 may comprise an acrylamide gel layer, or may be made from other materials including gelatin, agar, agarose, acrylamide, Sepharose®, Sephadex®, Sephacryl®, casein, unfixed gels and cross-linked gels. A thick gel layer 86 may be made from a mixture of materials. Gel layer 88 may be translucent, and is typically transparent to at least a portion of the electromagnetic spectrum, allowing imaging or scanning of small regions 90 within it.

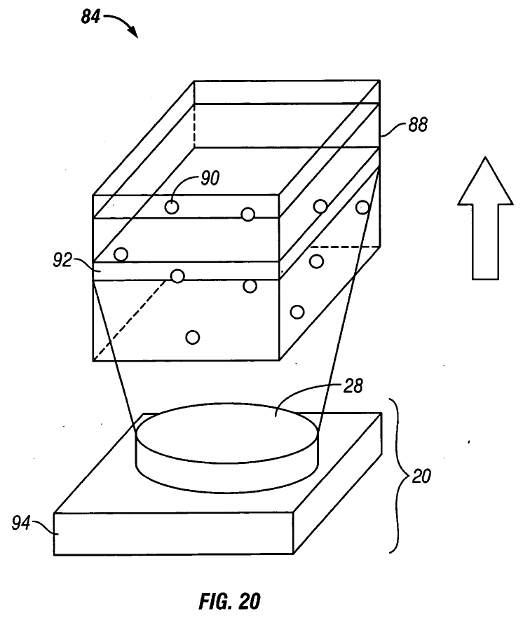

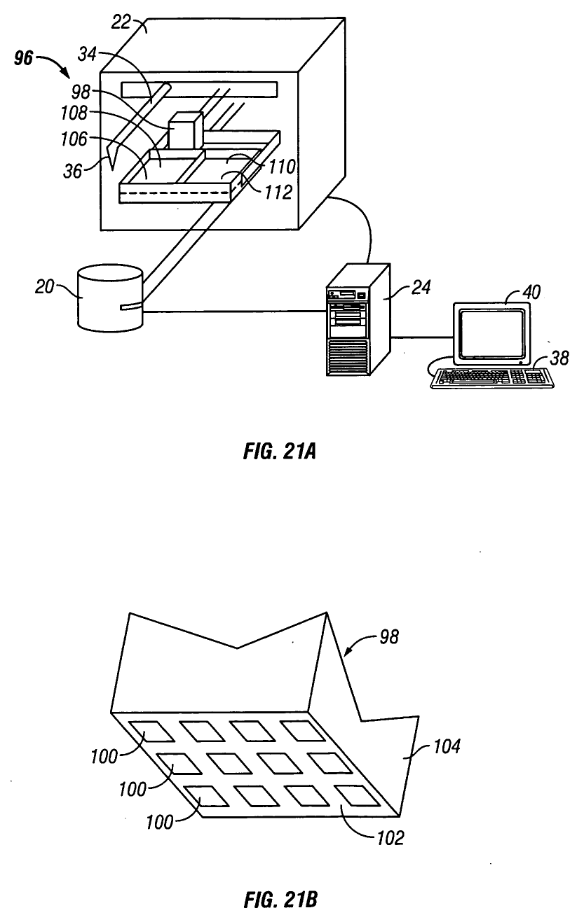

FIG. 21A illustrates a system 96 including a post 98 having wells 100 separated by hydrophobic regions 102. Wells 100 are configured to hold materials and reagents for PCR reactions, such as probes 14, primers 16, target nucleic acid molecules 18, and solutions for PCR. As shown in FIG. 21B, hydrophilic wells 100 of a post 98 are disposed at one end of the post 98, and are surrounded by a hydrophobic surface 102 which forms a border around the wells 100. A post 98 may be formed of a single piece of material, or may be made from a combination of materials (e.g., may be formed of a bundle of optical fibers). Wells 100 are typically formed of, or coated with, a hydrophilic material or a material that is not hydrophobic. Hydrophobic regions 102 may be exposed regions of the post material where that material is hydrophobic, or may be made from a coating of hydrophobic material placed onto the post material. Suitable materials for hydrophobic regions 102 comprise glass, plastic, hydrophobic metal, hydrophobic polymers, and other hydrophobic materials. Glass or other materials may be treated or coated to be hydrophilic (e.g., glass may be silanized to become more hydrophilic) and so would be suitable for use in making or coating wells 100. In some embodiments, target nucleic acid molecules 18 may be dispersed within a well 100 at a density of less than about ten target nucleic acid molecules 18 per cubic micron, or less than about one target nucleic acid molecule 18 per cubic micron, or less than about 10⁻¹ target nucleic acid molecules 18 per cubic micron, or less than about 10⁻² target nucleic acid molecules 18 per cubic micron, or less than about 10⁻³ target nucleic aci d molecules 18 per cubic micron, or less than about 10⁻⁴ target nucleic acid molecules 18 per cubic micron, or less than about 10⁻⁵ target nucleic acid molecules 18 per cubic micron, or less than about 10⁻⁶ target nucleic acid molecules 18 per cubic micron.

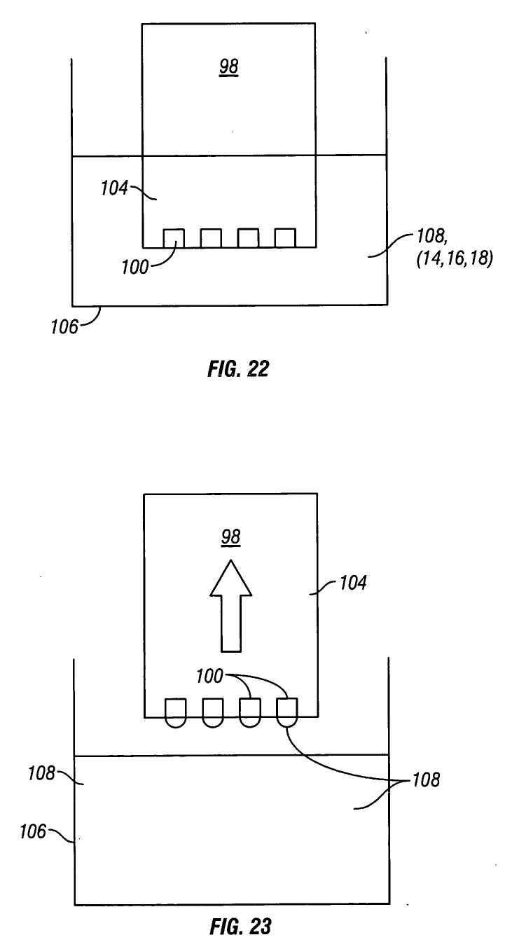

The PCR solutions including amplification and wash solutions are typically water-based solutions so that upon contact with wells 100 and hydrophobic regions 102, such solutions will tend to remain in contact with wells 100 and will tend to drain or flow away from hydrophobic regions 102. As illustrated in FIG. 22, the end 104 of a post 98 may be dipped into a trough 106 containing a water-based solution 108 containing a mixture of target molecules 18, probes 14, and primers 16. FIG. 23 illustrates that, upon removal of the end 104 of post 98 from the solution 108, wells 100 remain filled with the solution mixture 108, but that the hydrophobic surface 102 does not retain the solution 108 when the end 104 of the post 98 is extracted from the trough 106.

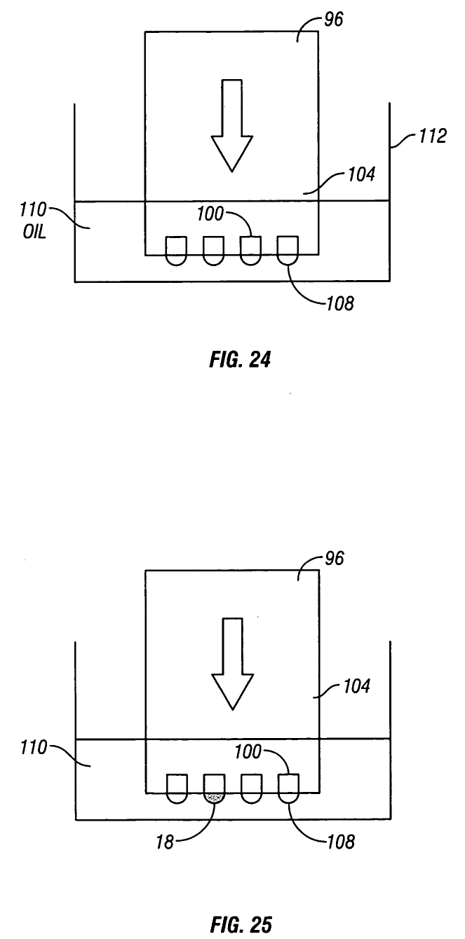

FIG. 24 illustrates the immersion of post 98 immersed into oil 110 (a hydrophobic solution) contained in a trough 112. Trough 112 for holding a hydrophobic solution such as oil 110 may be the same trough as is used to contain a hydrophilic solution 108 or may be a separate trough. Immersion of the end 104 of post 98 after filling wells 100 with a water-based solution 108 containing probes 14, primers 16 and target nucleic acid molecules 18 into oil 110 allows the mixture 108 to be thermal cycled without evaporation. For example, trough 112 may have or be connected with a heater or temperature controller for raising temperature for thermal cycling. A trough 112 may also have or be connected with a cooling element or controller. Thus, a system 96 including a post device 98 may be used to perform PCR on a solution 108 containing a mixture of target molecules 18, probes 14, and primers 16 effective to amplify and/or detect target molecules 18. Oil 110 may be mineral oil, silicone oil, petroleum-based oil, or other hydrophobic liquid.

FIG. 25 illustrates the amplification and fluorescence of probes 14 matching the target nucleic acid molecules 18 while end 104 of post device 98 is immersed in oil 110 effective to maintain separation of amplified copies of target nucleic acid molecules 18 and of indicators released from probes 14 during PCR cycles. Such separation is maintained by the isolation of separate aliquots of solution mixture 108 within wells 100. The oil and the hydrophobic regions 102 are effective to maintain this separation and to prevent or reduce evaporation of water from solution mixture 108 during thermal cycling. Such evaporation could be detrimental to the progress and accuracy of PCR, leading to undesired concentration of solutes, possible cessation of the PCR reaction in one or more wells 104, and other undesired effects.

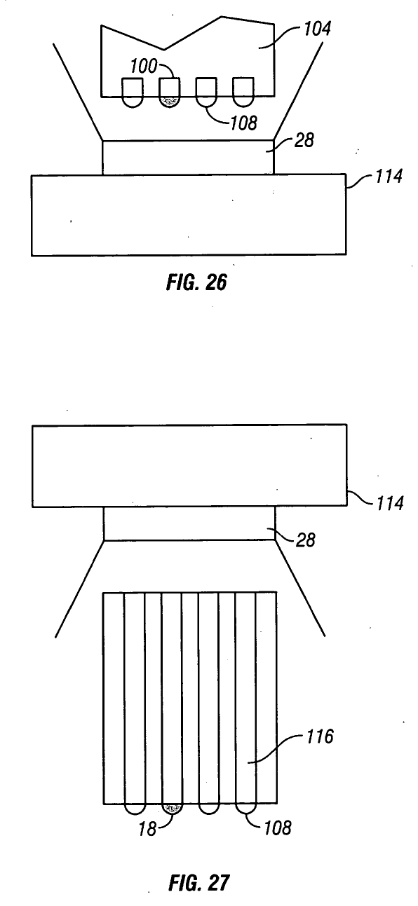

Following a desired number of thermal cycles, the end 104 of post 98 is removed from the oil 110 and may be inspected by eye or imaged to detect the presence of and/or quantify amplification of target nucleic acid molecules 18. FIG. 26 illustrates the post end 104 removed from oil 110 and imaged by a camera 114 with lens 28 to detect and count the fluorescence in wells 104. Imaging is typically by optical methods, and may be performed by camera, scanner, microscope (e.g., confocal microscope), or other suitable means.

FIG. 27 illustrates an alternative embodiment in which post device 98 comprises fiber optical bundles 116 configured to transmit optical radiation (such as infrared, visible, or ultraviolet light) to and from at least some of wells 100. A camera 114 or other detector may be used to image or detect fluorescence from wells 100 through a fiber optical bundle 116 in or on a post device 98. Camera 114 may have, or be operatively connected with, a light source or other source of electromagnetic radiation.

In some embodiments, a post device 98 may have wells 100 having depths or less than about 10 mm, or of less than about 5 mm, or less than about 2 mm, or of less than about 1 mm. Thus, wells 100 of a post device 98 may have depths of between about 5 mm and about 10 mm, or between about 2 mm and about 5 mm, or between about 1 mm and about 2 mm, or of between about 0 mm and about 1 mm. Wells 100 of a post device 98 may have widths of less than about 5 mm, or less than about 2 mm. A hydrophobic region 102 may be made of, or covered with, for example, a hydrophobic material such as plastic, hydrophobic polymers, glass, metal, or other hydrophobic material. A well 100 may be made or, or covered with, for example, a hydrophilic material such as silanized glass, polysaccharides, hydrophilic polymers such as polyester terephthalate (PET) and glycol-modified polyethylene terephthalate (PETG).

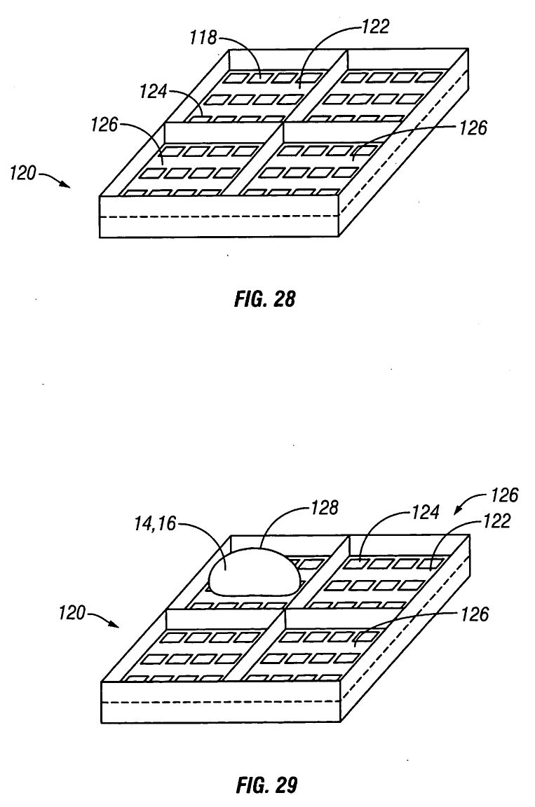

In an alternative embodiment, a hydrophobic surface configured as the bottom of a container for holding a solution may have multiple hydrophilic holes or depressions on the surface, forming hydrophilic wells separated by hydrophobic surfaces. For example, as illustrated in FIG. 28, hydrophilic depressions 118 are present in a plate 120 separated by hydrophobic surface 122 that has shallow walls 124 separating the plate 120 into wells 126. As illustrated, each well 126 has multiple hydrophilic depressions. Alternatively, a well 126 my have only a single hydrophilic depression.

As illustrated in FIG. 29, probes 14 and primers 16 may be added to a well 126 by deposition of a water-based solution 128. The probes 14 and primers 16 are then dried in a well 126. Drying may be accomplished, for example, by application of heat, or by illumination by a heating lamp, or at room temperature, in air, or under an inert gas such as nitrogen or argon, or by other suitable method. Multiple wells 126 may be prepared at one time, with solutions 128 placed in a well 126. Where a water-based solution 128 placed in one well 126 differs from a solution 128 placed in other wells 126 (e.g., different probes 14 and/or primers 16 may be used to make different solutions 128), each well 126 may then be prepared to detect and or amplify different target nucleic acid molecules 18. Alternatively, each well 126 may be prepared in the same manner as other wells 126. In some embodiments, target nucleic acid molecules 18 may be dispersed within a well 126 at density of less than about ten target nucleic acid molecules 18 per cubic micron, or less than about one target nucleic acid molecule 18 per cubic micron, or less than about 10⁻¹ target nucleic acid molecules 18 per cubic micron, or less than about 10⁻² target nucleic acid molecules 18 per cubic micron, or less than about 10⁻³ target nucleic acid molecules 18 per cubic micron, or less than about 10⁻⁴ target nucleic acid molecules 18 per cubic micron, or less than about 10⁻⁵ target nucleic acid molecules 18 per cubic micron, or less than about 10⁻⁶ target nucleic acid molecules 18 per cubic micron.

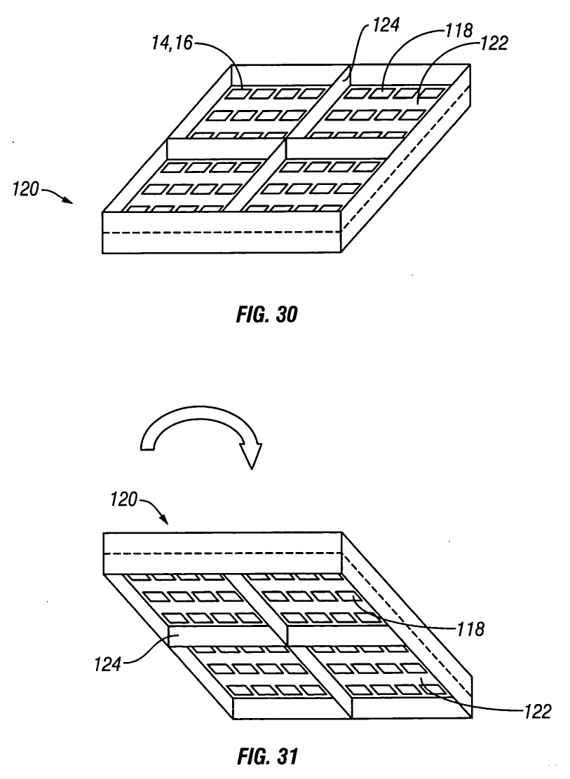

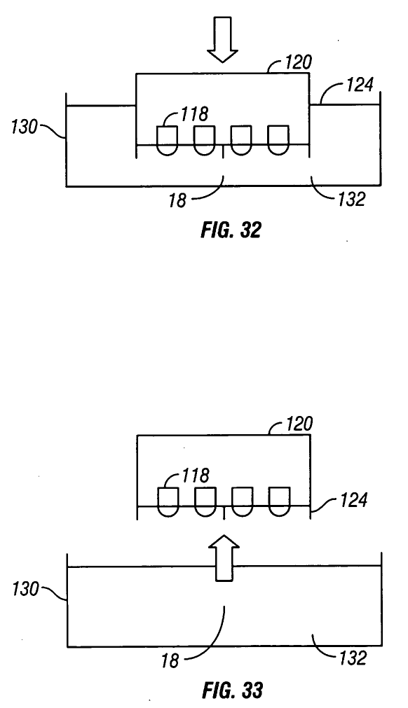

Probes 14 and primers 16 dried in a well 126 are illustrated in FIG. 30. Following drying, the plate 120 may be inverted, as shown in FIG. 31, and the plate with dried probes 14 and primers 16 inserted into a large trough 130 containing a target solution 132 (FIG. 32). Target solution 132 is a water-based solution containing target nucleic acid molecules 18 and may also comprise buffers, salts, or other constituents useful for PCR. FIG. 33 illustrates the plate 120 extracted from the large trough 130 out of target solution 132. Target nucleic acid molecules 18 are thus collected and mixed in target solution 132 with the probes 14 and primers 16 that were dried in the hydrophilic depressions 118.

The shallow walls 124 are effective to prevent the spread of probes 14 or primers 16 between wells 126 upon deposition of a solution 128 into a well 126. Thus, where different probes 14 or primers 16 are present in different wells 126, different assays may be performed simultaneously on a single plate to detect different target nucleic acid molecules 18 to which probes 14 and/or primers 16 are directed. Hydrophobic surfaces 122 also prevent the spread of solution between hydrophilic depressions 118, so that, after PCR, indicator dye or other means for identifying the presence of a target nucleic acid molecule 18 present in one hydrophilic depression 118 does not spread to a neighboring hydrophilic depression 118.

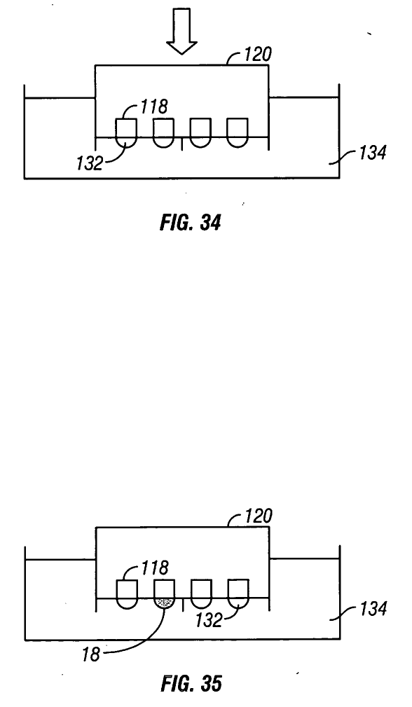

The plate 120 containing mixed probes 14, primers 16 and target nucleic acid molecules 18 in the hydrophilic depressions 118, may be inserted into oil 134 contained in an oil trough 136, allowing the mixture of targets 18, probes 14, and primers 16 to be thermal cycled without evaporation (FIG. 34). The target nucleic acid molecules 18 in one hydrophilic depression 118 are separated from those in another hydrophilic depression 118. Solution concentrations and PCR conditions may be adapted so that as few as a small number of target nucleic acid molecules 18, or a single of target nucleic acid molecule 18, may be found in any one hydrophilic depression 118 in a well 126. The number of copies of target nucleic acid molecules 18 increases with each PCR cycle, at least in the absence of depletion of primers 14, probes 16, or other necessary component of the PCR reaction. Diffusion of target molecules 18 away from their initial site within a hydrophilic depression 118 is limited by hydrophobic surface 122 between hydrophilic depressions 118. Thus, amplified copies of target nucleic acid molecules 18 derived from a single, or from a small number of, target nucleic acid molecules 18 will be found in hydrophilic depressions 118 within wells 126.

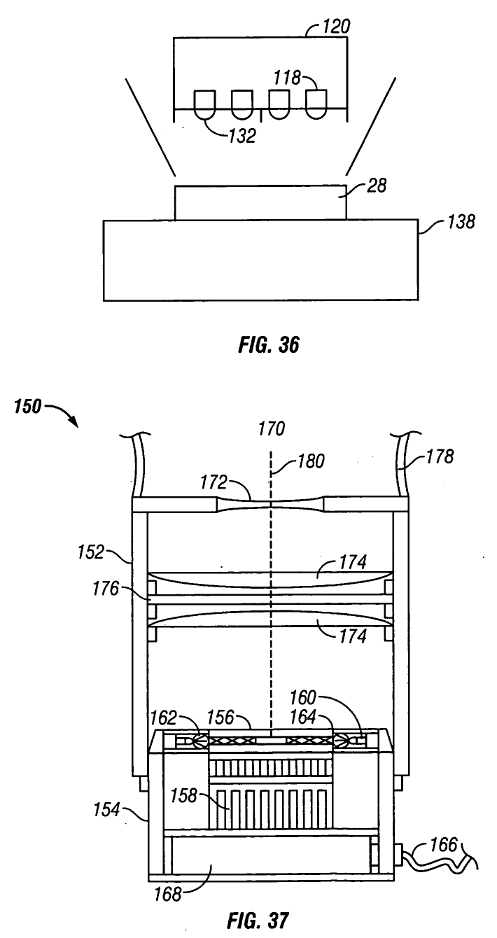

As illustrated in FIG. 35, after thermal cycling for amplification by PCR, presence of target molecules 18 is indicated by fluorescence derived from probes 14 matching the target nucleic acid molecules 18. Fluorescent indicator dyes may be released from probes 14, e.g., by degradation by amplification of target nucleic acid molecules 18 by elongation of primers 16 after amplifying by PCR. In some embodiments, the indicator dye may be derived from a Taqman® probe for use in a PCR system to indicate amplification of target molecules 18 recognized by probes 14 and primers 16. Such fluorescence may be detected (e.g., by eye, by camera, or other means) and/or quantified (e.g., counted), for example, by camera imaging with a camera 138 of the plate 120 as illustrated in FIG. 36.

In some embodiments, hydrophilic depressions 118 may have depths of less than about 10 mm, or of less than about 5 mm, or less than about 2 mm, or of less than about 1 mm. Thus, hydrophilic depressions 118 may have depths of between about 5 mm and about 10 mm, or between about 2 mm and about 5 mm, or between about 1 mm and about 2 mm, or between about 0 mm and about 1 mm. Hydrophilic depressions 118 may have widths of less than about 10 mm, or less than about 5 mm, or less than about 2 mm. Wells 126 may have lengths and widths of up to about 5 cm, or of up to about 2 cm, or of up to about 1 cm or less. Thus, wells 126 may have lengths and widths of between about 2 cm and about 5 cm, or between about 1 cm and about 2 cm, or less than about 1 cm.

Also provided are methods for amplifying and/or detecting small numbers of nucleic acid molecules. Methods herein comprise contacting a first solution having probe and/or primer molecules with a channel, or chamber, effective that the solution is conducted along the channel or into the chamber, the probe and/or primer molecules being configured to hybridize with at least a portion of a target nucleic acid molecule. Solution flow may be by capillary action, although it may be produced or aided by pressure or suction. The solution is dried so that the primer and/or probe molecules are retained within the channel or chamber. A second solution containing a target nucleic acid molecule is contacted with the channel or chamber so that the second solution is conducted along the channel, or into the chamber, effective to mix the target nucleic acid molecule, dried probe and primer molecules into the second solution at an initial temperature. The initial temperature may be, for example, room temperature. Where the first solution has probes but not primers, the second solution may include primers. Where the first solution has primers but not probes, the second solution may include probes. Alternatively, where the first solution has only one of probes and primers, but no both probes and primers, the other reagent (primers or probes) may be provided by a third solution, included with the sample, or by other means.

Valves may be used to regulate the flow of one or more solutions. For example, valves may be polyethylene glycol (e.g., of a molecular weight selected to have the desired melting temperature and degradation characteristics), wax, polymer, or other material which may be removed or altered to allow passage of material by e.g., heat, or light, electrical power, or other controlling signal. In some embodiments of the methods, barriers may be raised or placed to limit diffusion along the channels after target nucleic acid molecules have passed along them.

Heat is then applied to raise the temperature of the mixed target nucleic acid, probe and primer molecules, separating nucleotide dimers, and then the temperature is allowed to become reduced, completing a thermal cycle. PCR amplification is allowed to occur, and then the temperature is raised again, to separate complementary stands. Upon reduction of temperature, amplification of target nucleic acid molecules again occurs, and the process is repeated a sufficient number of times effective to produce nucleic acid copies of said target nucleic acid molecule effective to amplify and/or detect the target nucleic acid molecules.

In some embodiments of methods disclosed herein, the second solution comprising a target nucleic acid molecule is a dilute solution containing a plurality of target nucleic acid molecules, effective that individual target nucleic acid molecules are separated from one another within the channel. It will be understood, however, that solutions may be applied in any order, and that designation of a first solution and of a second solution is not meant to limit the order of application of solutions in methods disclosed herein. In some methods, indicators are cleaved from probes by the extending primers to generate fluorescence around targets that match the probes. The indicators may be fluorescent indicators. In some methods, Taqman® probes are used. Multiple indicators, such as fluorescent dyes with different fluorescence wavelengths, may be used to indicate different target molecules or different conditions.

Thermal cycles may be repeated after a short interval of time so as to prevent diffusion of target nucleic acid molecule copies greater than a short distance from the individual target nucleic acid molecules that were copied. The number of cycles may be greater than about fifty, or fewer than about fifty, or fewer than about thirty, or fewer than about twenty cycles, or may be fewer than about ten cycles. The second solution may be, but need not be, a viscous buffer solution.

In some embodiments of the methods, amplification of the target nucleic acid molecule is detected by detection of an optical signal, such as a fluorescence signal. In some embodiments of the methods, detection is effected as soon as possible after the last thermal cycle in order to limit the distance that nucleic acid copies or indicators travel away from their point of origin or release. Viscous buffers may also be used to reduce or slow the diffusion of nucleic acid copies and indicator molecules. Detection may be by eye, and may be by optical methods, using, for example, optical devices such as cameras, scanners, microscopes, such as a confocal microscope, charge-coupled devices, and photomultiplier tubes. Multiple signals (such as multiple fluorescent wavelengths) may be detected, simultaneously or sequentially (e.g., by the use of multiple detectors simultaneously, or by a single detector and multiple filters used sequentially). Detection of indicators may be ongoing during the amplification process, or may be performed at intervals during the process. For example, fluorescence measurements may be taken soon after the completion of each thermal cycle.

In further methods, a small number of nucleic acid molecules may be amplified and/or detected by contacting a first solution containing a primer molecule and a probe molecule with a gel within a well and allowing at least some of the primers and probes to diffuse into the gel. A second solution containing a target nucleic acid molecule is contacted with the gel, so as to mix together the target nucleic acid molecule with probe and primer molecules within the gel. The mixing occurs at an initial temperature. Heat is then applied to raise the temperature of the mixed target nucleic acid, probe and primer molecules to a raised temperature above the initial temperature. Target nucleic acid dimers will dissociate at raised temperatures. The temperature is then allowed to become reduced to a temperature closer to the initial temperature. Amplification of the target nucleic acid molecules by PCR occurs. The temperature is then raised, and PCR thermal cycles are repeated effective to produce nucleic acid copies effective to amplify and/or detect the target nucleic acid molecule. In some methods, the thermal cycles are repeated more than about thirty times, or less than about thirty times, or less than about twenty times, or less than about ten times.

Suitable PCR methods include 5′ nuclease methods, such as Taqman® methods, using probes having fluorescent indicators such as dyes that may be cleaved from probes during primer extension. In the practice of the methods disclosed herein, the gel may be a thin gel layer, which may have a thickness of less than about 1 mm, or less than about 0.5 mm, or a less than about 0.1 mm, or less than about 0.05 mm. Thus, the gel may be a thin gel layer having a thickness of between about 0.5 mm and about 1 mm, or between about 0.1 mm and about 0.5 mm, or between about 0.05 mm and about 0.1 mm, or between about 0 mm and about 0.05 mm. In some embodiments, the thin gel layer comprises a gel layer having a thickness of about 0.04 mm.

In some embodiments of methods, a thick gel layer is contacted with a solution containing primer molecules, probe molecules and target nucleic acid molecules. The gel is preferably within a well. The probe, primer and target molecules are allowed to diffuse into the gel at an initial temperature. Heat is then applied to raise the temperature of the mixed target nucleic acid, probe and primer molecules to a raised temperature above the initial temperature. Target nucleic acid dimers will dissociate at raised temperatures. The temperature is then allowed to become to a temperature closer to the initial temperature. Amplification of the target nucleic acid molecules by PCR occurs. The temperature is then raised, and PCR thermal cycles are repeated effective to produce nucleic acid copies effective to amplify and/or detect the target nucleic acid molecule. In some methods, the thermal cycles are repeated more than about fifty times, or less than about fifty times, or less than about thirty times, or less than about twenty times, or less than about ten times.

Another embodiment of the methods for amplifying and/or detecting small numbers of nucleic acid molecules comprises placing a hydrophilic well that is surrounded by a hydrophobic surface in contact with a solution containing a target nucleic acid molecule, a primer molecule and a probe molecule. Removing the hydrophilic well the solution allows a portion of the solution to remain in contact with the hydrophilic well, while substantially no solution remains in contact with the hydrophobic surface. This provides separate aliquots of solution in the separate hydrophilic wells. Placing a portion of the device having the hydrophilic wells into a hydrophobic liquid, such as an oil, so that the solution within the hydrophilic well remains in contact with that well. Application of PCR, including thermal cycling, is then performed to make copies of the target nucleic acid molecule within the solution in the well. Copies are detected, indicating the presence of the target nucleic acid molecule and its amplification. For example, fluorescence from indicators cleaved from probes as the copies are made may be detected by optical methods, and the presence and amount of target nucleic acid determined. Detection of electromagnetic radiation, such as infrared, visible, or ultraviolet light, may be by, e.g., a detector (such as a camera, charge-coupled device, photomultiplier, or other optical device) and may be by use of one or more optical fibers.

A further method for amplifying and/or detecting small numbers of nucleic acid molecules comprises applying a first solution containing a primer molecule and a probe molecule in contact with a hydrophilic well surrounded by a hydrophobic surface. The first solution is dried so that probe and primer molecules dry into or onto at least a portion of the hydrophilic well. The hydrophilic well is then contacted with a volume of a second solution containing a target nucleic acid molecule, and the well removed from contact with the volume of second solution, leaving a mixture of target nucleic acid molecules, probes, and primers in the second solution in contact with the well. At least a portion of the substrate is placed into contact with a hydrophobic liquid, such as an oil, so that the mixture remains in contact with the well. The oil is effective to prevent escape or dispersal of the second solution from the well. Copies of the target nucleic acid molecule are then made using PCR techniques within or adjacent the mixture. The presence of the target nucleic acid molecule or copies of it are then detected. Progress of the PCR amplification is detected and monitored, an may be detected by optical methods. Taqman® probes and techniques may be used. Alternatively, the order of application of the solutions may be reversed, with or without drying some elements prior to application of other elements.

Fluorescent indicators suitable for use in some embodiments of the methods comprise fluoroscein dyes (U.S. Pat. Nos. 5,188,934; 6,008,379; 6,020,481), rhodamine dyes (U.S. Pat. Nos. 5,366,860; 5,847,162; 5,936,087; 6,051,719; 6,191,278), benzophenoxazine dyes (U.S. Pat. No. 6,140,500), energy-transfer dye pairs of donors and acceptors (U.S. Pat. Nos. 5,863,727; 5,800,996; 5,945,526), cyanines (Kubista, WO 97/45539), ethidium bromide, propidium iodide, and other fluorescent molecules. Examples of fluorescein dyes comprise 6-carboxyfluorescein; 2′,4′,1,4,-tetrachlorofluorescein; and 2′,4′,5′,7′,1,4-hexachlorofluorescein (Menchen, U.S. Pat. No. 5,118,934).

The term “substrate” refers to a base surface which may support other elements or surfaces of a device. A substrate may be, in part or wholly, composed of metal, glass, plastic, ceramic, or other material. A substrate may be, for example, glass, silica, quartz, controlled-pore-glass (CPG), or reverse-phase silica. A substrate may comprise such materials as oligosaccharides, nitrocellulose, diazocellulose, dextran, agar, agarose, Sepharose®, Sephadex®, Sephacryl®, cellulose, starch, nylon, latex beads, magnetic beads, paramagnetic beads, superparamagnetic beads, and microtitre plates. Plastics, such as organic polymers, may comprise, for example, polyacrylamide, polycarbonate, polyimide, polymethylmethacrylate, polydimethylsiloxane, polyethylene, polyethyleneoxy, polyfluoroethylene (including polytetrafluoroethylene), polypropylene, polysulfone, polystyrene, polypropylene, polyurethane, and polyvinylchloride, as well as co-polymers and grafts thereof.

A substrate may be translucent (allowing the passage of optical radiation) or transparent (allowing the passage of optical radiation with little loss or distortion), or may have a portion that is translucent or transparent (e.g., a window). Translucent or transparent materials comprise glass, quartz, polycarbonate, polymethylmethacrylate, and other materials.

A substrate may form or support a well, depression or other container, vessel, feature or location. A substrate may have features such as channels, grooves, pathways, wells, barriers, or other features effective to contain a fluid and to direct and control the flow of fluid. Such features may be fabricated in a solid substrate by any suitable method, including molding (e.g., injection molding). Alternatively, or in addition, such features may be fabricated by microfabrication techniques such as lithographic techniques used in fabrication of semiconductor devices, (including, for example, photolithographic etching, plasma etching, and wet chemical etching). Such features may be fabricated by micromachining techniques such as laser drilling or laser ablation, micromilling, air abrasion, LIGA, reactive ion etching, embossing, and other techniques known in the art. LIGA (an acronym based on the first letters for the German words for lithography and electroplating) is a well-known process for fabricating features and devices with very small dimensions. A general review of the LIGA process is given in the article by W. Ehrfeld, et al., “LIGA Process: Sensor Construction Techniques Via X-Ray Lithography,” Technical Digest IEEE Solid State Sensor and Actuator Workshop, 1988, pp. 14-4. Fabrication methods suitable for preparing embodiments of devices described herein are described, for example, in U.S. Pat. Nos. 5,162,078, 5,378,583, 5,527,646, 5,631,514, 5,679,502, 5,571,410, 5,917,260, and 6,176,962 d and references cited therein.

Fluid flow within a microfluidic device is typically directed by the walls of a channel configured to contain the fluid. Design of such channels is described in, for example, U.S. Pat. No. 5,842,787 and references cited therein. Flow within a small capillary or thin gel layer may be by capillary action. Solutes within a fluid may flow by diffusion. Pathways configured for fluid flow may be treated or coated to reduce adsorption of solutes flowing within them. For example, a channel may be silanized or may be coated with bovine serum albumin (BSA), cytochrome C, or other protein or chemical to reduce non-specific adsorption of protein, nucleic acid, or other material to the walls of the channel. A fluid flowing within a channel may itself contain BSA for the same purpose.

Fluid flow may also be effected by pressure gradients, temperature gradients, voltage gradients, osmotic gradients, or by other means or combination of means. A pressure gradient may be provided by a pump, or by compression of all or part of a chamber or channel, or in other ways. Alternatively, fluid flow may be impelled by electrophoresis, or by electro-osmotic means. Movement of the device (e.g., rotation to create centrifugal force) may also be used to impel or direct fluid flow. Thus, hydraulic, electrokinetic, osmotic, or other means may be used to direct fluid flow along or within a channel or other such structure. Flow may be regulated or stopped by a valve, or a gate or barrier, or in other ways.

A substrate may have a plurality of locations, and be configured to define locations in an array. The various locations may be addressable for robotic delivery of reagents, or for detection of hybridization at the locations. Detection of hybridization may be by eye, and may be by detection means including camera, photomultiplier, scanning by laser illumination and confocal or deflective light gathering, or by other means.

A detector may be a device or system for sensing a signal provided by a target or indicator. A detector may be configured, for example, to detect radiation, fluorescence, phosphorescence, luminescence, pH, charge, current, voltage, redox potential, absorbance, temperature, and/or may comprise an electrical, magnetic, thermal, acoustic, or other sensor. A detector typically comprises an optical detector, such as, for example, a photomultiplier tube, a charge-coupled device (CCD), a scanning detector, a confocal device, or other device.

The methods, devices, assemblies and systems disclosed herein enable highly-quantitative counting of single copy events using Taqman® reagents and other assays for amplification of target nucleic acid molecules. Methods, devices, assemblies and systems disclosed herein are further illustrated in the following EXAMPLES which illustrate assemblies, devices, and methods for use in systems having features of the invention. Nucleic acid targets are distributed in such low concentration that single copies can be amplified and fluoresce without diffusing with other targets. In this way, as few as only a single copy of target can be amplified a million fold in a very small volume. For example, using Taqman® assay methods each target copy will generate an unquenched reporter. The reporters fluoresce when illuminated by a light source. A means of low noise excitation coupled with the highly concentrated signal enables detection by eye, avoiding the need for expensive photon sensors and optics. However, if desired, photon sensors and optics may be used in place of, or in addition to, observation by eye.

The methods, devices, assemblies and systems described above and in the Examples below, may be used to detect the presence of target nucleic acids indicative of the presence of target organisms. Such target organisms include pathogens, such as bacterial, viral, fungal, or other pathogens. A target pathogen may be, e.g., Mycobacterium Tuberculosis or Bacillus anthracis, which cause tuberculosis and anthrax, respectively. Thus, for example, a target pathogen may be one that causes tuberculosis, anthrax, diphtheria, meningitis, whooping cough, tetanus, pneumonia, rabies, influenza, smallpox, or other disease. Such pathogens may indicate disease in a sample taken from a host animal or a human patient or from a bodily fluid or waste from a host animal or a human patient. Detection of target nucleic acid from a target organism in a sample of food, raw material, effluent, water source, material used in the production of a pharmaceutical or of an industrial product, or other material may also be used to detect the presence of a target organism in the source of the sample.

For example, gram positive and gram negative bacteria can be detected. Gram positive bacteria to be detected include, for example, bacteria belonging to the genera Staphylococcus, Streptococcus, Listeria, Clostridium, and Corynebacteria. Gram negative bacteria to be detected include, for example, bacteria belonging to the family Enterobacteriaceae. Gram negative bacteria to be detected include, for example, gram negative bacteria belonging to the genera Haemophilus, Bacteroides, Pseudomonas, Neisseria, and Legionella can be detected. Target organisms to be detected may include fungi belonging to the genera Candida, Cryptococcus, Coccidiodes and Histoplasma. Viral pathogens may also be detected, e.g., viruses from Paramyxoviridae, Rhabdoviridae, Filoviridae, Boma Disease Virus, Orthomyxoviridae, Bunyaviridae and Arenaviridae, including viral pathogens such as nucleic acids derived from, and indicative of, influenza, herpes, polio, smallpox, hepatitis, human immunodeficiency virus (HIV), Ebola, hanta, or other viruses. Where the virus to be detected is an RNA virus (e.g., the virus has genetic material encoded by ribonucleic acid (RNA)), reverse transcriptase enzymes may be included with the reagents used so as to provide deoxyribonucleic acid (DNA) copies of the viral nucleic acid.

### EXAMPLE 1

The human eye is a very sensitive detector, capable of detecting as few as 10 photons landing within a an area of about 50 micrometer in diameter. An example of a device having features disclosed herein in which the human eye may serve as a detector is shown in FIG. 37. Such a device may be termed, for example, a digital Taqman® reader. The device 150 shown in this example consists of three assemblies: optical assembly 152, thermal assembly 154, and reaction assembly 156. The assemblies are configured to fit together to form an operative assemblage or device comprised of the assembled component assemblies. An optical assembly 152 may include, for example, an aperture, a lens, an optical filter, and other optical elements. A thermal assembly 154 may include, for example, a heating element, a cooling element, or a heating and cooling element, a controller to control the operation of such thermal elements, a power source, and other elements. A reaction assembly 156 may include, for example, a reaction chamber for reacting a solution including a target nucleic molecule with assay reagents suitable for detecting (e.g., by hybridization) and for amplifying the numbers of, the target nucleic acid molecule. The device 150 may be configured for assembly and for dis-assembly, for example, to allow removal and replacement of a used reaction assembly 156 by a fresh reaction assembly 156 for use with assemblies 152 and 154 in an assembled device 150 after re-assembly.

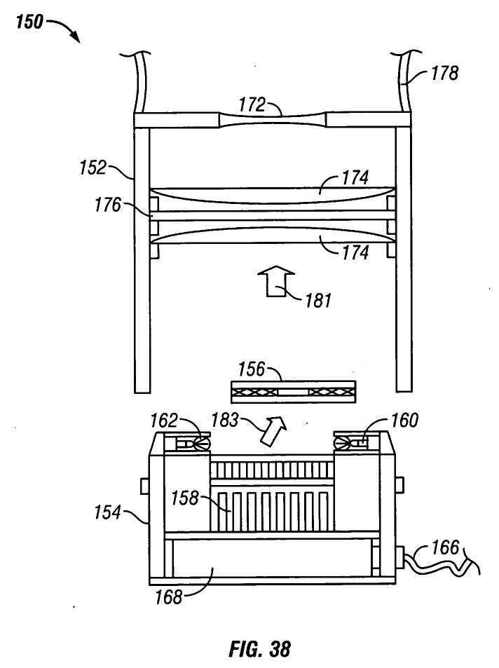

A thermal assembly 154 may be configured to receive a reaction assembly 156, e.g., to hold a reaction assembly 156 in contact with thermal elements of the thermal assembly 154. As illustrated in FIGS. 37 and 38, a reaction assembly 156 can be mounted into a thermal assembly 154, making physical contact with a thermal cycler unit such as a peltier device 158. A light source 160 (which may include multiple light sources 160) can illuminate the reaction assembly 156. Illumination may be via a lens, or lenses 162 as shown. Illuminating through the edge 164 of the reaction assembly 156 is a particularly efficient means of illumination. Many light source types may be used (including, e.g. light emitting diodes (LEDs), lasers, lamps or other light sources) and small lenses 162 may be used to focus the light into the reaction assembly 156. External power 166 or batteries can power the thermal cycler 158 and light source 160, and other components as needed as well. A controller 168, such as an electronic controller, may be used to control the temperature, cycle time, light source activation, and may be used to initiate notification of the operator when to look into the optics (e.g., by means of a buzzer, flashing light, or other prompt) and may perform other functions as well.

A detector 170 is used to detect light indicating the progress of the assay reactions and to detect the presence of target nucleic acid molecules and copies of such molecules. The human eye can be the detector 170, but a camera, photo-multiplier tube, a charge-coupled device, or other detector could also be used in addition to, or in place of, the eye of a human observer. Light is detected by a detector 170 via aperture 172 and may be aided by a lens or lenses 174 and a filter or filters 176. An aperture 172 may be an opening, or may be a window (e.g., a transparent or translucent covering across an opening), and may be an opening that may be covered by a shutter, a diaphragm, or other element. Where the detector 170 is a human eye, an eye-guard 178 may be helpful to protect the eye of the observer. An eye guard 178 may be a flexible eye-guard 178, and may assist in blocking external light from the eye. An eye guard 178 may also serve to position the eye of an observer, and may serve to attach other forms of detectors 170 as well.

The optical assembly 152 is designed to mechanically couple onto the thermal assembly 154, blocking external light and aligning its optical axis 180 to the reaction assembly 156. As shown, the optical assembly 152 may contain several lenses 174 (which may be positive and/or negative lenses) or none at all, depending on the light collection efficiency needed to detect an assay in the reaction assembly 156. A filter 176 or filters 176 may also be needed, particularly long pass filters 176 to block out light from the excitation source while passing the longer wavelength fluorescence. Arrow 181 indicates light traveling form the reaction assembly 156 towards lenses 174 and filter 176.

A device 150 as disclosed herein may be configured to be reusable, or to be disposable, and different portions or assemblies may individually be configured either for re-use or to be disposable. For example, the device 150 illustrated in FIG. 37 may be configured so that the reaction assembly 156 comprises a disposable assembly, and the optical assembly 152 and thermal assembly 154 are configured to be used more than once. The arrow 183 in FIG. 38 indicates removal of reaction assembly 156 from contact with thermal assembly 154. FIG. 38 shows three component assemblies (optical assembly 152, thermal assembly 154, and reaction assembly 156) dis-assembled, enabling the removal of a used reaction assembly 156 (as shown in FIG. 38) and insertion of a new reaction assembly 156 (not shown).

Assemblies as illustrated in FIGS. 37 and 38 may be produced for a reasonably low cost. For example, components in the optics and thermal assemblies (e.g., optical filters and lenses, light sources such as light-emitting diodes (LEDs), heater/cooling assemblies such as peltier devices, power supplies and controllers) are readily available in production quantities from commercial suppliers.

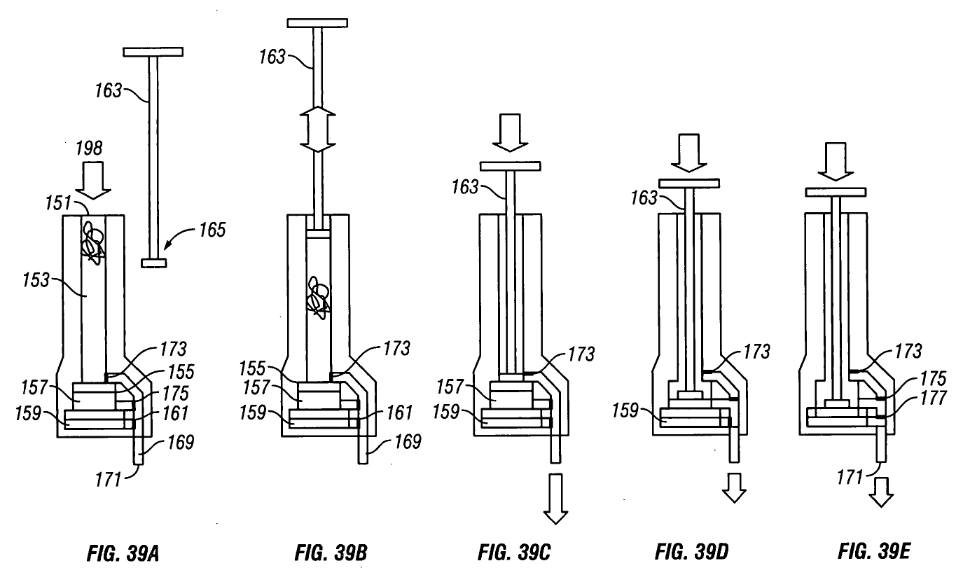

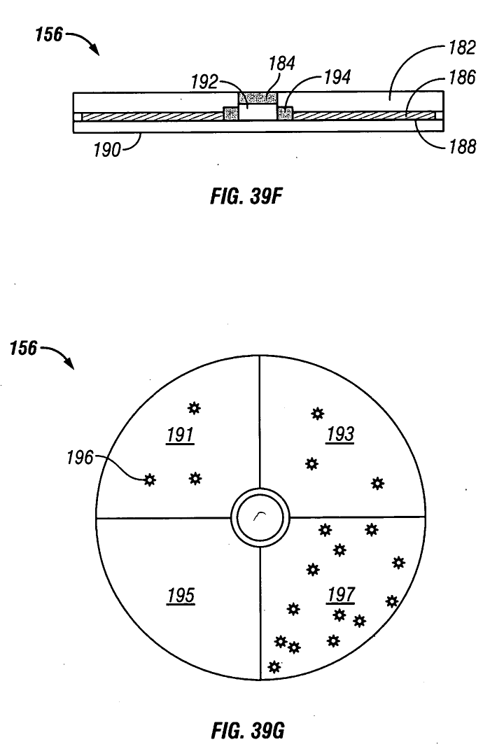

A sample may be introduced directly into a reaction assembly 156, or may be processed prior to introduction into a reaction assembly 156. FIGS. 39A-39E illustrate steps in one method of preparing a sample 198 for delivery to a device 150 with a reaction assembly 156. A sample 198 is introduced into a chamber 151 containing a lysing buffer 153 containing enzymes suitable for lysing a cell or virus (e.g., proteolytic, lipolytic and/or glycolytic enzymes). A valve 155 separates the lysing buffer 153 from a wash buffer 157; wash buffer 157 is separated from elution buffer 159 by valve 161. Placement of piston 163 into chamber 151, and moving distal portion 165 of piston 163 inwardly and outwardly within chamber 151 is effective to mix sample 198 with lysing buffer 153. Movement of piston 163 for a brief time such as, for example, 30 seconds, is effective to lyse cells present in sample 198 mixed in lysing buffer 153 and to release cellular contents into solution (indicated by checked pattern shown in FIG. 39B). Following mixing and lysing, opening valve 173 and depressing piston 163 further into chamber 151 forces lysing buffer 153 containing sample 198 through channel 167 containing silica beads 169. Silica beads 169 capture DNA released from cells in sample 198 while allowing passage of lysing buffer 153 out of output port 171. Subsequent opening of valve 155, opening of valve 175 to allow wash buffer 157 to pass into channel 167, and further depression of piston 163 forces wash buffer 157 over and past beads 169 to remove any remaining lysing buffer 153. Subsequent opening of valve 161, opening of valve 177 to allow elution buffer 159 to pass into channel 167, and depression of piston 163 forces elution buffer 159 over beads 169 removing DNA that had been adherent to beads 169, providing a processed sample 198. Processed sample 198 may thus flow from output port 171 under the influence of piston 163 when desired for further analysis or for detection of target nucleic acid molecules present in sample 198.

FIGS. 39F and 39G show the reaction assembly 156 in greater detail. The side view (FIG. 39F) shows components of the reaction assembly 156 including a window 182, septum 184, filter 194, reagent chamber 186 filled with pre-deposited reagents 188, and a thermally conductive support 190. Pre-deposited reagents may dried-down onto a surface or surfaces of the reagent chamber 186, or may be otherwise deposited in place within a reagent chamber 186. Such reagents may include, for example, one or more of nucleic acid probes, nucleic acid primers, nucleotide triphosphates (e.g., deoxynucleotide triphosphates (dNTPs) for amplification of DNA), polymerase enzymes, magnesium, other salts, buffers, and other agents. A port 192 covered by the septum 184 and bounded by a filter 194 provides access to the reagent chamber 186 (which may be, for example, where the reagents used are Taqman® reagents, a Taqman® chamber). In FIGS. 39F and 39G, and in all figures, it will be understood that features that appear multiple times in an illustration, and symmetric features that appear, e.g., on each of two sides of a central feature may be labeled only one time, or only on one side of the figure, while the label is to be understood to apply to all the corresponding features in the illustration. For example, in FIG. 39F, reagent chamber 186, with reagents 188 deposited therein, is illustrated on both sides of septum 184 and port 192, although the figure labels reagent chamber 186 and reagents 188 only on one side of the figure.

The top view (FIG. 39G) shows how the location of individual copies of genomic DNA (gDNA) from a pathogen is detected at a location 196 (indicated by stars) as a bright fluorescent spot. The presence of a pathogen is indicated by the presence of one or more bright fluorescent spots. The number of genomic copies (number of pathogens) can be counted to indicate the degree of sample contamination by the pathogen. Different target nucleic acid molecules may be detected in single reaction assembly 156 by providing different probes and primers configured to recognize different target nucleic acid molecules. As indicated by the numbers 191, 193, 195 and 197 in each of the four quadrants of reaction assembly 156 illustrated in FIG. 39G, different probes and primers that recognize different target nucleic acid molecules may be localized to different locations within a reaction assembly 156. In embodiments, different probes and primers that recognize different target nucleic acid molecules may be localized to the same location within a reaction assembly 156, and may be distinguished, for example, by having different fluorescent molecules as indicators.

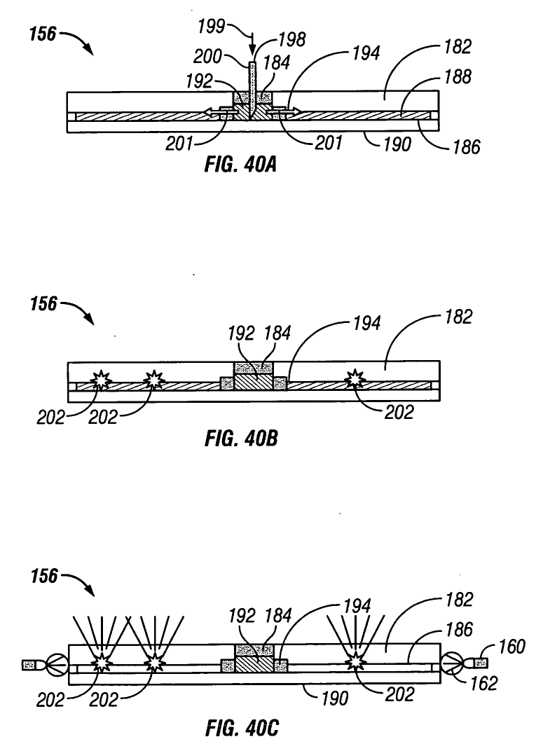

FIGS. 40A, 40B, and 40C show the operations of an assay in a reaction assembly 156. The user loads a liquid sample 198 into the reaction assembly 156 by puncturing the septum 184 with a needle 200 and injects the liquid sample 198 into the port 192 (as indicated in FIG. 40A by the downward-facing arrow 199). A very small distance (e.g. ≦100 um) separates the window 182 and support 190 such that when sample 198 is injected into the port 192, capillary force immediately draws the liquid sample 198 into the reagent chamber 186, but the filter 194 blocks large cell debris from entering the reagent chamber 186 (flow of sample 198 into reagent chamber 186 is indicated by horizontal arrows 201 in FIG. 40A). In this way, sample 198 that has been added to reaction assembly 156 is drawn into the reagent chamber 186. All reagents 188 needed for the reaction (e.g., a Taqman® reaction) are pre-deposited in the reagent chamber 186, including DNA probes, DNA primers, dNTPs, polymerase enzymes, magnesium, and salts. By spreading out the sample 198, even a single target of interest is isolated into a small volume with all the reagents 188 required for amplification, reducing the probability that a higher concentration of other DNA will inhibit its amplification.

After the reagent chamber 186 is filled with sample 198, the reaction assembly 156 is thermal cycled (FIG. 40B). Temperatures used for thermal cycling may vary between reactions used, but typically range from about 50° C. to about 75° C. for the lower temperatures to about 80° C. to about 99° C. for the higher temperatures. Typical thermal cycling temperatures for Taqman® reactions are a high of about 95° C. and a low of about 65° C. Ideally, each thermal cycle will double the number of any targeted gDNA sequence 196 (e.g., from a pathogen present in the sample 198), so twenty thermal cycles could generate over a million copies of a specific GDNA sequence 196. In the process of duplication, a fluorescent molecule (reporter 202 indicated by stars in FIG. 40B) is separated from a quenched probe sequence when reagents match a gDNA present in the sample 198, such that the number of free reporters 202 equals the number of target amplifications. During amplification, these reporters 202 are free to diffuse in the reagent chamber 186, but fast thermal cycling limits the diffusion distance to a few 100 micrometers. Thus, if the user immediately activates a light source 160 after thermal cycling, a large number of reporters 202 fluoresce (indicated by lines radiating upwardly from reporters 202 in FIG. 40C) in a very small volume will generate a very bright spot 196 (FIG. 40C). If the gDNA targets are few, they will likely be distinctly separated in the reagent chamber 186, enabling the user to count the number of gDNA by eye. Although in the simplest case, no optics are necessary, in most cases lenses 174 will be useful to focus the signal to aid its detection by a detector 170.

### EXAMPLE 2

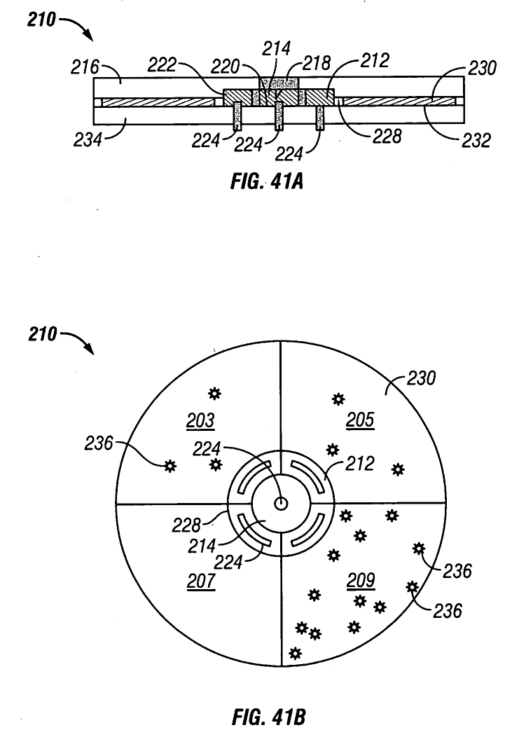

FIGS. 41A and 41B show a reaction assembly 210 containing multiple sample preparation chambers buffer chamber 212 and lysing chamber 214. It will be understood that corresponding features in FIGS. 41A and 41B (and similarly for other figures having multiple members) have the same meanings in each figure, even where the feature is labeled in only one of the two figures. The side view (FIG. 41A) shows components of the reaction assembly 210 including a window 216, septum 218, lysing chamber 214 filled with pre-deposited lysing reagents 220, buffer chamber 212 filled with pre-deposited buffer 222, electrodes 224, separating matrix 226 (e.g. a perforated membrane, grid, scintered partition, or other matrix), valve 228, reagent chamber 230 filled with pre-deposited (e.g., dried-down) reagents 232, and a thermally-conductive support 234. Lysing reagents 220 may include enzymes such as proteloytic, lipolytic, and glycolytic enzymes, and/or other agents suitable for lysing cells to release cellular contents into solution, or to attack a viral particle to release viral nucleic acids. Reagents 232 may include, for example, one or more of nucleic acid probes, nucleic acid primers, nucleotide triphosphates (e.g., deoxynucleotide triphosphates for amplification of DNA), polymerase enzymes, magnesium, other salts, buffers, and other agents. A reagent chamber 230 may include, for example, the reagents suitable for a Taqman® essay. A valve 228 may be, e.g., a time valve set to open at a set time, or after a set interval has passed after a triggering event. The top view (FIG. 41B) shows the locations 236 of genomic DNA (gDNA) from the sample 238. A single copy of genomic DNA from a pathogen is amplified and can be detected as a bright fluorescent spot at a location 236. The number of genomic copies (number of pathogens) can be counted to indicate the degree of sample contamination. As indicated by the numbers 203, 205, 207 and 209 in each of the four quadrants of reaction assembly 210 illustrated in FIG. 41B, different probes and primers that recognize different target nucleic acid molecules may be localized to different locations within a reaction assembly 210. In embodiments, different probes and primers that recognize different target nucleic acid molecules may be localized to the same location within a reaction assembly 210, and may be distinguished, for example, by having different fluorescent molecules as indicators.

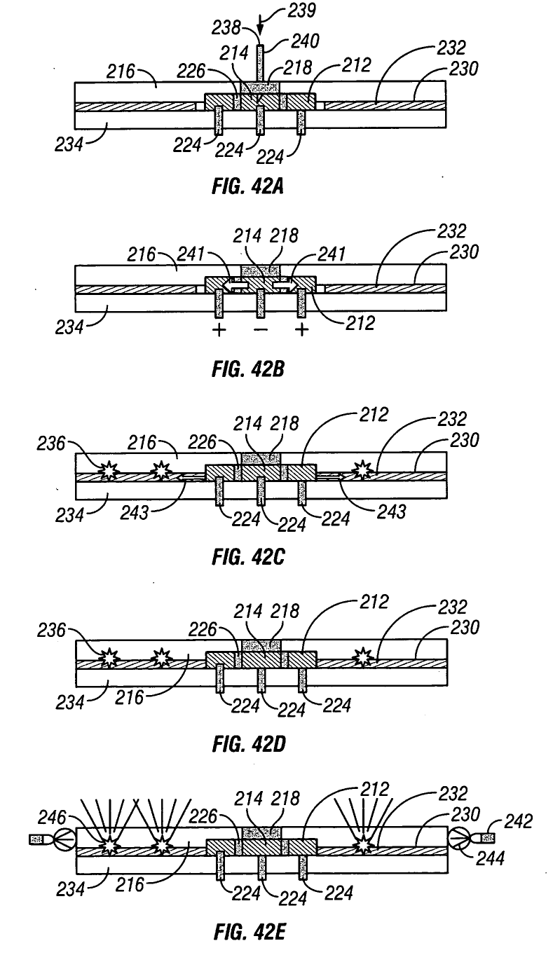

The reaction assembly 210 illustrated in FIGS. 42A, 42B, 42C, 42D, and 42E is configured to be used with other elements such as the optical assembly 152 and thermal assembly 154, for example, as illustrated in FIGS. 37 and 38 and may be used in place of a reaction assembly 156 described above. FIGS. 42A, 42B, 42C, 42D, and 42E show the operations in a reaction assembly 210. The user loads crude sample 238 into the reaction assembly 210 by puncturing the septum 218 with a needle 240 and injecting the liquid sample 238 into the lysing chamber 214 (injection is indicated in FIG. 42A by the downward arrow 239). Injection of the sample 238 into the lysing chamber 214 of the reaction assembly 210 mixes the sample 238 with reagents 220 which have been pre-deposited in lysing chamber 214. Pre-deposited lysing reagents 220 lyse cells present in the crude sample 238, releasing any pathogen gDNA that maybe present into free solution. The liquid sample being conductive, addition of the sample 238 also closes an electrical circuit by providing electrical connectivity between electrodes 224. Closure of the circuit is effective to initiate, or to signal to initiate, application of a voltage gradient between electrodes 224 within sample 238 and to initiate electrophoresis of the gDNA from the lysing chamber 214. The voltage gradient is effective to electrophorese the GDNA through the separating matrix 226, and into the buffer chamber 212 (FIG. 42B). Electrophoresis is indicated by the horizontal arrows 241 to indicate material crossing matrix 226 from lysing chamber 214 into buffer chamber 212. Power and control for such electrophoresis may be provided, e.g., by a controller 168 and power 166 as illustrated in FIGS. 37 and 38. Activation of the electrodes 224 is indicated in FIG. 42C by the symbols + and − under the electrodes 224, which also indicate the polarity of the voltage gradient imposed by the voltage between the electrodes 224.

Cell debris and chemicals that do not electrophorese, and particles larger than the pores of matrix 226 will not enter the buffer chamber 212. The prevention of the entry of such cell debris, chemicals, and particles prevents the inhibition of PCR that might otherwise occur in the presence of such cell debris, chemicals, and particles. After sufficient time for most or all of the gDNA to be electrophoresed into the buffer chamber 212, the valves 228 open. Opening of valves 228 allows passage of material into reagent chamber 230 (as indicated by horizontal arrows shown in FIG. 42C). Target gDNA is indicated by stars 236. Valves 228 may be time-valves, in which opening is controlled by a timer or the passage of time. Valves 228 may be opened by dissolving, melting, mechanical action, electrical current, or by other means, and control of the opening of valves 228 may be by timers, by sensors, by a controller 168, may be manually controlled, or may be controlled by other means. Thus, valves 228 may be mechanical valves, such as gates, that are able to open, or may be made of material that degrades, changes state or is removed, so that by its degradation, change, or removal a pathway between buffer chamber 212 and reagent chamber 230 is provided. Thus, for example, valves 228 may be polyethylene glycol (e.g., of a molecular weight selected to have the desired melting temperature and degradation characteristics), wax, polymer, or other material.

In embodiments, a very small distance (e.g. ≦100 μm) separates the window 216 and the support 234 such that when the valves 228 open, capillary force draws the buffer 222 (including the gDNA from sample 238) from the buffer chamber 212 into the reagent chamber 230, displacing air in the reagent chamber 230 into the buffer chamber 212. Entry of sample solution including nucleic acids into reagent chamber 230 is indicated by arrows 243. All reagents 232 needed for the Taqman® reaction or other desired amplification reaction are available in the reagent chamber 230. Reagents 232 may be provided in the reagent chamber 230 by being pre-deposited in the reagent chamber 230 or may be provided by being pre-deposited in the buffer chamber 212 (from where they are drawn into the reagent chamber 230 by capillary action). Reagents 232 include, e.g., DNA probes, DNA primers, dNTPs, polymerase enzymes, magnesium, other salts, buffers, and other agents.

After the reagent chamber 230 is filled with sample 238, the reaction assembly 210 is thermal cycled (illustrated in FIG. 42D). Progress of PCR or other assay reaction unquenches reporter molecules when reagents match or otherwise detect target gDNA in the sample. Unquenched reporter molecules fluoresce upon illumination from a light source. Immediately after thermal cycling, the user activates the light source 242 (which may be directed towards the reagent chamber 230 with a lens or lenses 244) and counts the number of bright spots 246 detectable though window 216 as a direct measurement of the number of pathogens in the sample (FIG. 42E).

### EXAMPLE 3

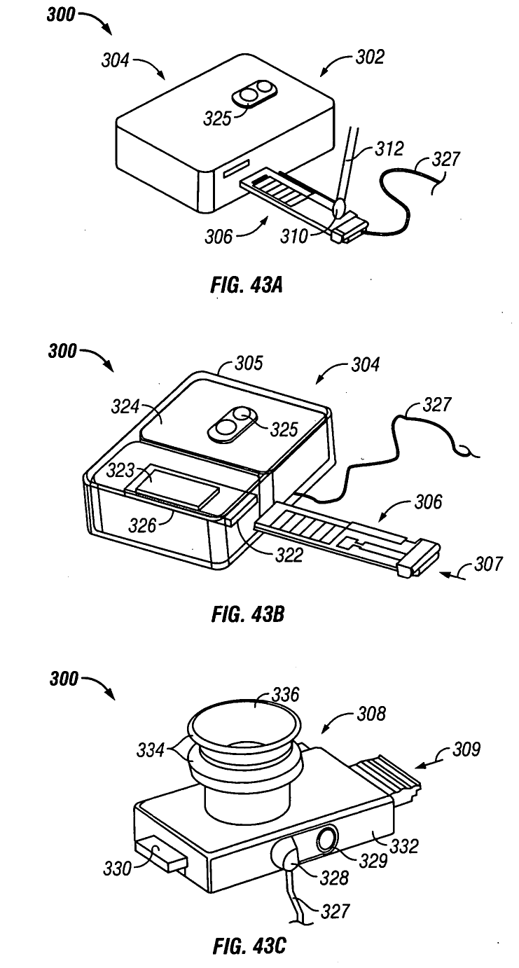

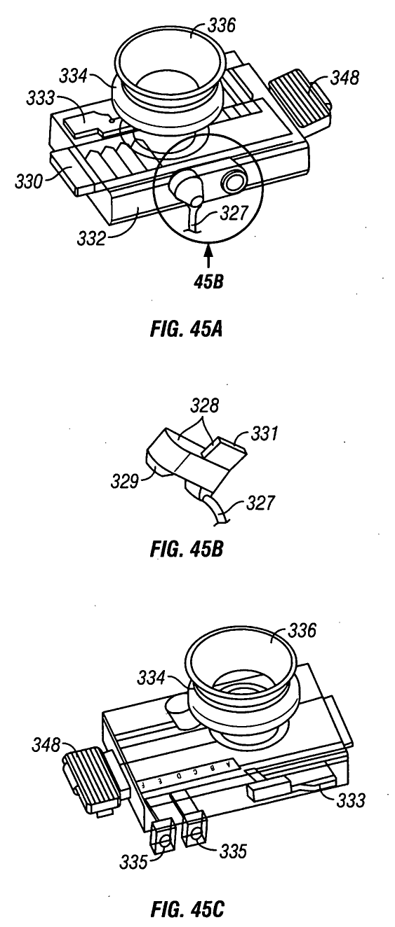

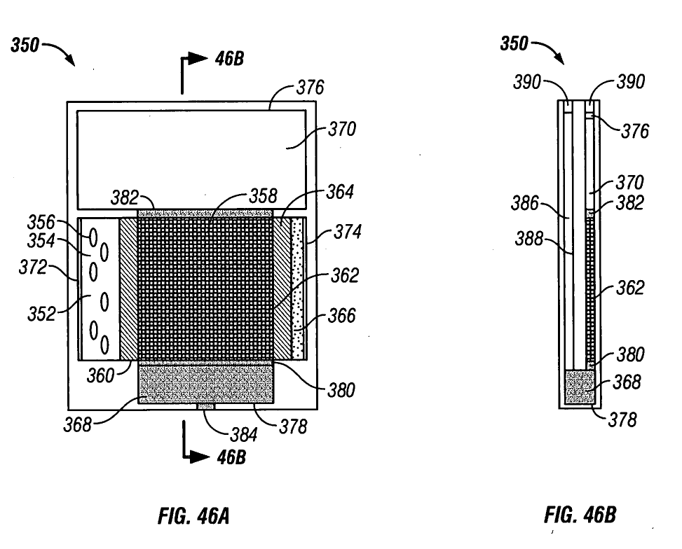

A further embodiment of devices and systems having features of the invention is illustrated in FIGS. 43-48. For example, FIG. 43A illustrates application of a sample to an assay cartridge, and illustrates a thermal assembly, of an analytical assembly of a system having features of the invention. According to the methods and apparatus disclosed herein, as illustrated in FIG. 43A, 43B and 43C, a system 300 may comprise an analytical assembly 302 comprising a thermal assembly 304 and an assay cartridge 306. A system 300 may further include a detection assembly 308. As shown in FIGS. 43A, 43B and 43C, a sample 310 may be added by pipette 312 to an input port 314 of an assay cartridge 306 for analysis and possible detection of a target nucleic acid molecule (e.g., gDNA of a target pathogen that may be present in a sample). Input port 314 is connected to a preparation chamber 316, which may comprise part or all of preparation module 318 configured for preparing sample 310 for delivery to one or more assay chambers 320. For example, as illustrated in more detail in FIGS. 45 and 46, a preparation module 318 may include a cross-electrophoresis chromatography module configured to lyse cells in a sample 310, separate the contents of the lysed cells, and provide a processed cellular preparation for delivery to, and analysis in, assay chambers 320.

A thermal assembly 304 is configured to receive or engage an assay cartridge 306, e.g., within a slot 322 or other receiving element, socket or receptacle. Thermal assembly 304 includes a thermal source 323, such as a peltier device, capable of heating and/or cooling an assay cartridge 306 received within or engaged with a thermal assembly 304. In embodiments, multiple assay cartridges 306 may be received within a thermal assembly 304, sequentially and/or simultaneously. A thermal assembly 304 may also include a power source (e.g., a battery 324), an on/off switch 325, and a controller 326. In embodiments, a thermal assembly 304 may be connected to an external power source in addition to, or instead of, including a battery 324. A controller 326 is typically configured to control the timing and temperature of thermal cycling of the thermal source 323, and may perform other functions as well (e.g., provide and/or record signals related to status or progress of a thermal cycle, or of a series of cycles, or of other operations). A cable 327 may connect to a light source 328. A thermal assembly may further include other features, including indicators (e.g., numerical or alphabetic displays, and light-emitting diodes (LEDs) for signaling status, progress or completion of an operation, occurrence of an unexpected event or malfunction), connectors suitable for data transfer.

FIG. 43B is a perspective view of a thermal assembly 304 of a system having features of the invention, also indicating elements located within the outer casing 305 of the thermal assembly 304, also indicating elements located within the outer casing 305 of the thermal assembly 304. As illustrated in FIG. 43B, an assay cartridge 306 may be inserted into a slot 322 of a thermal assembly 304 following addition of sample 310 to assay cartridge 306. Insertion is indicated by the wide arrow 307. Such insertion may follow immediately, or soon after addition of sample 310 to assay cartridge 306, or may follow after an interval (e.g., an interval to allow sufficient time for the action of reagents on the sample 310 in input port 314 or preparation module 316), or after other operations on the sample 310.

Detection assembly 308 is a further element of a system 300. Detection assembly 308 is configured to engage and receive a light source 328, which, as illustrated in FIG. 43C, may be connected to thermal assembly 304 via cable 327 (from which it may receive power (e.g., from battery 324) and/or control). Such a light source 328 may be locally controlled (e.g., by a button 329 as shown in FIG. 43C) or may receive control signals from a distal source (e.g., from controller 326). Light source 328 is shown removably attached to detection assembly 308 in FIG. 43C; however, in embodiments, a light source 328 may be a permanent element of a detection assembly 308, and may receive power and control unrelated to a thermal assembly 304.

FIG. 43C illustrates insertion of the assay cartridge 306 shown in FIGS. 43A and 43B into slot 330 in a detection assembly 308 of a system 300 having features of the invention. Insertion is indicated by the wide arrow 309. In use, a thermal assembly 304 having a loaded assay cartridge 306 engaged within slot 322 will alternately heat and cool assay cartridge 306 in order to amplify target nucleic acid molecules that may be present in a sample 310. Insertion of assay cartridge 306 into slot 330 in casing 332 of a detection assembly 308 typically follows completion of thermal cycling in a thermal assembly 304, so that any target nucleic acid molecules present in an assay chamber 320 of an assay cartridge 306 will have been amplified and will be ready for detection. A detection assembly 308 as illustrated in FIG. 43C includes an observational assembly 334, including optics such as, e.g., lenses and filters, and a lip 336 which may serve as an eye guard (when detection is by eye) and/or mounting surface for mounting a camera, photodetector, or other sensing device whereby detection may be performed electronically or by other artificial means. Insertion of assay cartridge 306 into slot 330 places portions of assay cartridge 306 into position within detection assembly 308 effective to allow detection of target molecules. For example, as illustrated in FIG. 43C, insertion of assay cartridge 306 into slot 330 places assay chambers 320 into position within detection assembly 308 so as to allow observation and detection of any target molecules located in an assay chamber 320.

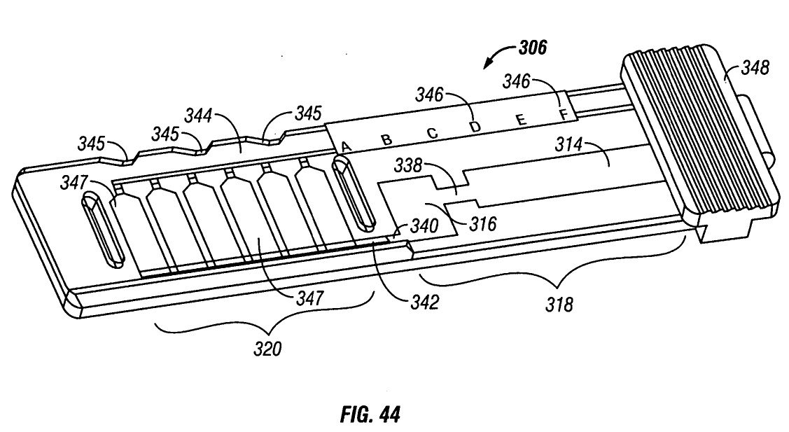

FIG. 44 illustrates features of an assay cartridge 306 in greater detail than shown in FIGS. 43A, 43B or 43C. Assay cartridge 306 having features of the invention includes a sample preparation module 318 and assay chambers 320. Sample preparation module 318 has an input port 314 configured to receive a sample 310, input port 314 being operatively connected to a preparation assembly 316 by a channel 338. Sample 310 is processed within preparation assembly 316 (e.g., may be mixed, filtered, separated, reagents may be added, cells lysed, and/or other actions taken) to aid in the reactions and analysis that take place in the assay chambers 320. Probes and/or primers 347 may be deposited in assay chambers 320 in readiness for use with sample 310 when it flows into assay chambers 320. In embodiments, different sets of probes and/or primers may be present in different assay chambers 320, so that one assay chamber 320 may be configured to amplify and/or detect, e.g., a first target nucleic acid, while a second assay chamber 320 may be configured to amplify and/or detect, e.g., a second target nucleic acid, within the same assay cartridge 306. Following such processing steps, sample 310, or portions thereof, may exit preparation assembly 316 via output port 340 into input channel 342 for delivery to one or more assay chambers 320. Output channel 344 provides an outlet for sample 310 for further processing or analysis, if desired, and may provide an exit route for air and for solution to be discarded.

An assay cartridge 306 as illustrated in FIG. 44 has mechanical features for positioning the assay cartridge 306 within a thermal assembly 304 and within a detection assembly 308. For example, notches 345 are effective to engage with positioning elements within a thermal assembly 304 or a detection assembly 308 to insure proper placement of the assay cartridge 306. Such proper placement includes, for example, placement of assay chambers 320 within a detection assembly 308 aligned properly with optical elements of a detection assembly 308. Indicators 346 are useful, for example, to display the particular position, of multiple possible positions, taken by an assay cartridge 306 within a thermal assembly 304 or detection assembly 308, and may be used to mark or indicate in which assay chamber a target molecule has been detected. Other mechanical features, such as handle 348, and including the size and shape of the assay cartridge, ridges, guides, slots, and other features may be effective to provide proper placement and engagement of an assay cartridge 306 with a thermal assembly 304 and detection assembly 308.

A detection assembly 308 engaged with an assay cartridge 306 is shown in FIGS. 45A and 45C. Illumination by light source 328 allows observation of assay chambers 320 aligned with optical elements of the observational assembly 334 as indicated in these Figures. Light source 328 is shown mounted on casing 332 in FIG. 43A. FIG. 45B provides a perspective view of light source 328 in greater detail, showing light source 328 when not mounted on detection assembly 308, allowing a view of light element 331 (typically an LED) which fits into position for illumination of assay chambers 320 when mounted on detection assembly 308. Button 329 may be used to initiate and/or control illumination by light element 331.

As shown in FIG. 45C, levers 333 may be used to position an assay cartridge 306 within a detection assembly 308. Assay chambers 320 are positioned so as to enable their inspection with observational assembly 334 for detection of target nucleic acid molecules from a sample 310. When positioned with particular assay chambers 320 aligned with optical elements of an observational assembly 334, indicators 346 are correspondingly aligned with buttons 335 so that the presence of a target nucleic acid in an assay chamber 320 may be indicated by the depression of a button 335 effective to mark an indicator 346 corresponding to the assay chamber 320 having the target nucleic acid molecule. Such marking may be indicated, for example, by highlighting, coloring, or otherwise inking the indicator, by scratching, scoring, or otherwise mechanically altering the indicator, by electrical means, or by other means.

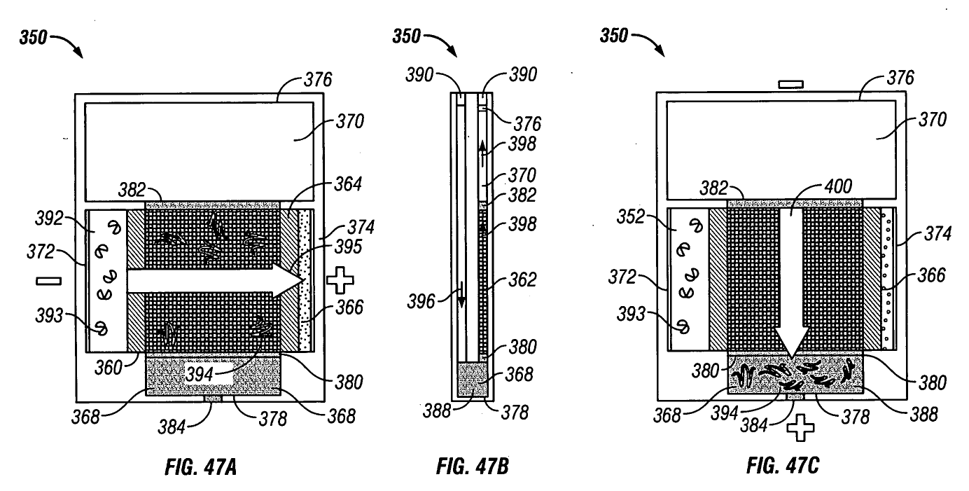

Sample preparation within a preparation module 316 is further illustrated in FIGS. 46 and 47, in which an embodiment of a integrated sample preparation module having features of the invention is shown. FIG. 46A shows a top view of a preparation module 350 having features of the invention that is configured to provide cross-electrophoresis chromatography. A cross-sectional view (taken along line 46B-46B of FIG. 46A) of the cross-electrophoresis chromatography preparation modules 350 shown in FIG. 46B. An input chamber 352 is configured to receive a sample 354. A sample 354 may be deposited into an input port 314, as shown in FIGS. 43 and 44, and then transported to an input chamber 352, or, alternatively, in embodiments, may be deposited directly into input chamber 352. A sample may include, e.g., cells 356. A matrix chamber 358 is positioned adjacent input chamber 352, separated from input chamber 352 by a matrix 360 configured to filter sample 354 so that debris, particles, and other non-essential elements are prevented from passing from input chamber 352 into matrix chamber 358. Matrix chamber 358 comprises a matrix 362 suitable for separation of cellular elements, e.g., suitable for electrophoresis or other chromatographic separation. A third matrix 364 forms a distal boundary of matrix chamber 358 effective to prevent egress of sample elements desired to remain within matrix chamber 358. First matrix 360 may include, for example, a gel such as agarose, and may include a filter, membrane, fiber, or other material suitable for impeding or blocking the passage of cellular debris. Second matrix 362 is configured to retain nucleic acid molecules, and may include silica, such as silica beads or silica fiber, and may include metal such as aluminum. Third matrix 364 may be similar to first matrix 360, e.g., may comprise a gel such as agarose, and may be configured to accept and to retain protein, so that proteinaceous materials from a sample 354 may flow towards waste chamber 366 and be at least partially prevented by third matrix 364 from returning to matrix chamber 358 or from flowing into output chamber 368.

As illustrated in FIGS. 46A and 46B, a first waste chamber 366 is located opposite the third matrix 364 for collection and ultimate disposal of waste elements from a sample 354. Output chamber 368 and second waste chamber 370 are also located adjacent matrix chamber 358 as shown in the figures. Electrodes 372, 374, 376 and 378 are configured to provide electric current and voltage effective for electophoresis of sample elements within the preparation module 350. Application of a voltage difference between any two of electrodes 372, 374, 376 and 378 is effective to apply a voltage gradient within the matrix chamber 358 between the electrodes. Fluid flow is regulated by valves 380, 382, and 384. Elution buffer chamber 386 contains elution buffer 388 and is connected to output chamber 368, as shown in FIG. 46B. Elution buffer 388 may simply be a wash solution, or may include reagents and components suitable for subsequent actions with the sample 354. For example, elution buffer 388 may include salts, buffers, and/or reagents suitable for use in performing PCR such as, for example, polymerase enzymes, dNTPs, and/or other reagents. Air vents 390 allow for airflow into and out of chambers so that fluid flow, by capillary action or by other means of providing fluid flow, is not impeded by changes in gas pressure within chamber. A preparation module 350 as illustrated in FIGS. 46A and 46B may be of any suitable size. For example, a preparation module 350 may have dimensions on the order of about one or a few millimeters (mm) to about several centimeters (cm) or more. In embodiments, a preparation module 350 may be about 10 to about 50 mm wide, about 10 to about 50 mm long, and about 2 to about 20 mm deep, where width is represented as the left to right dimension in FIG. 46A, length is represented as the up to down dimension in FIG. 46A, and depth is represented as the left to right dimension in FIG. 46B. In embodiments, a preparation module 350 may be about 20 mm wide, about 25 mm long and about 6 mm deep.

Operation of a cross-electrophoresis preparation module 350 is illustrated in FIGS. 47A, 47B and 47C. Sample 354 within input chamber 352 is mixed with lysis buffer 392, causing lysis of cells 393 and release of cellular contents. Cellular contents, but not particles or cellular debris, pass across first matrix 360 into matrix chamber 358 having second matrix 362. Application of voltage between first electrode 372 and second electrode 374 is effective to electrophorese nucleic acid 394 from lysed cells 393 of sample 354, separating and capturing nucleic acid 394 within second matrix 362. Arrow 395 indicates the direction of the electrophoretic flow of nucleic acid 394 under the influence of the voltage imposed by electrodes 372 and 374 (the polarity of the voltage is indicated by plus and minus symbols shown in the Figures). Waste material flows through third matrix 364 and into waste chamber 366, while nucleic acid 394 is substantially retained by second matrix 362. During the electrophoresis procedure illustrated in FIG. 47A, first valve 380 and second valve 382 are closed, further acting to constrain flow along the direction indicated by arrow 395.

Following the electrophoresis illustrated in FIG. 47A, voltage is no longer applied between electrodes 372 and 374, and lysis buffer 392 is washed from matrix chamber 358. First valve 380 and second valve 382 are opened, allowing flow of elution buffer 388 by capillary action within buffer chamber 386 and into matrix chamber 358. The direction of buffer flow within buffer chamber 386 is indicated by arrow 396; the direction of elution buffer flow within matrix chamber 358 is indicated by arrows 398. Elution buffer 388 displaces lysis buffer 392 within matrix chamber 358, washing lysis buffer 392 into waste chamber 370.

FIG. 47C is a top view schematic of a preparation module 350 illustrating a later processing step. Following flow of lysis buffer 392 out of, and flow of elution buffer 388 into matrix chamber 358, first valve 380 is opened and a voltage is applied across matrix chamber 358 by imposition of a voltage difference between the third electrode 376 and the fourth electrode 378 effective to electrophorese nucleic acid molecules 394 into output chamber 368. Arrow 400 indicates the direction of electrophoretic flow of nucleic acid molecules 394 within matrix chamber 358; the polarity of the voltage is indicated by the plus and minus signs adjacent electrodes 376 and 378. In this way, output chamber 368 contains nucleic acid molecules 394 in elution buffer 388 substantially free of cellular debris or particles, and substantially free of other elements not needed or that might interfere with subsequent assays on the nucleic acid 394 from sample 354.

Opening third valve 384 allows nucleic acid molecules 394 in elution buffer 388 to flow out of output chamber 368 for analysis. For example, an output chamber 368 may be connected to input channel 342 and assay chambers 320 as illustrated in FIGS. 43 and 44 effective to direct a sample 354 (after processing) into an assay chamber 320.

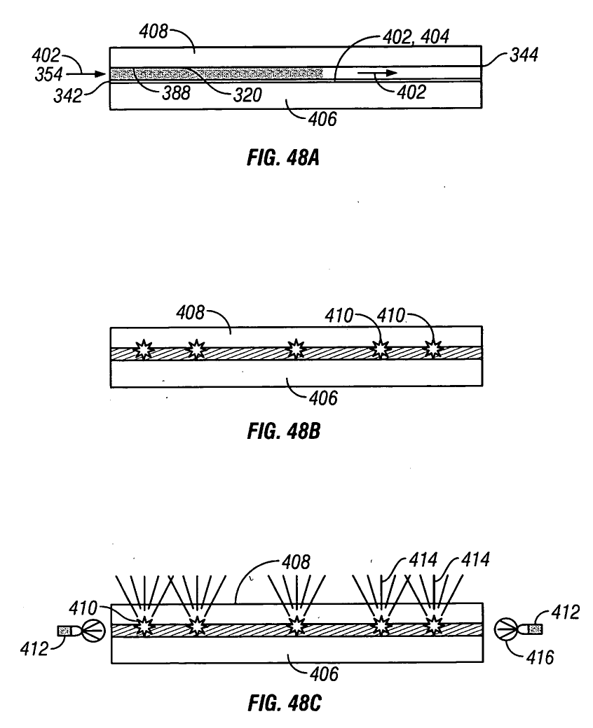

As illustrated in FIGS. 48A, 48B and 48C, an assay chamber 320 is suitable for performing assay reactions, e.g., PCR reactions. In embodiments, an assay chamber 320 may have pre-positioned probes and/or primers ready for combination with elution buffer 388 and nucleic acid molecules 394 after processing of a sample 354 in other portions of a system 350. As discussed above, for example, probes and/or primers may be pre-deposited (for example, by inflow of a solution containing the probes and/or primers into an assay chamber 320, and subsequently drying down the solution to leave the probes and/or primers deposited on a surface or surfaces within an assay chamber 320). Other reagents may also be pre-deposited, or may be introduced by solution flow (e.g., contained within elution buffer 388) after introduction of sample 354 into input chamber 352. Such reagents include, for example, nucleotide triphosphates (e.g., deoxynucleotide triphosphates (dNTPs) for amplification of DNA), polymerase enzymes, magnesium, other salts, buffers, and other agents. The assay chamber 320 shown in schematic side view in FIG. 48A is configured to draw nucleic acid molecules 394 and elution buffer 388 into chamber 320 by capillary action. Combination of elution buffer 388 containing salts, buffers, and/or amplification reagents (e.g., PCR reagents) with probes and/or primers that have been previously introduced within an assay chamber 320 provides conditions suitable for performance of PCR within an assay chamber 320. For example, as shown in FIG. 48A, with the direction of flow indicated by arrows 402, nucleic acid molecules 394 flow with elution buffer 388 from input channel 342 into assay chamber 320 (shown in cross section). PCR probes and/or primers 404 directed to a target nucleic acid molecule are present within assay chamber 320. In embodiments, the upper surface 408 of the assay chamber 320 is translucent or transparent, allowing passage of fluorescent, transmitted or reflected light for observation of signals from a location or locations within assay chamber 320, e.g., for detection of target nucleic acid molecules. The lower surface 406 of assay chamber 320 may be transparent or translucent, or may be opaque. Air within an assay chamber 320 may flow out an output channel 344 as elution buffer 388 and nucleic acid molecules 394 low into assay chamber 320. Nucleic acid molecules 394, elution buffer 388, and probes and/or primers 404 mix within assay chamber 320, so that assay chamber 320 contains all reagents necessary for the desired amplification reaction (e.g., PCR). Where only probes or only primers are present within an assay chamber 320 prior to inflow of nucleic acid molecules 394 and elution buffer 388, the other amplification agent (e.g., probes where primers are already present, or primers where probes are already present) may be provided in the elution buffer 388, or may introduced with another solution, or by other means.

Following introduction of nucleic acid molecules 394 and reagents, assay chamber 320 and its contents are subjected to thermal cycling effective to amplify nucleic acid molecules 394 present in sample 354 within assay chamber 320. Amplified target nucleic acid molecules 410 shown in FIG. 48B may have been amplified by PCR or other amplification methods. Illumination of the assay chamber 320 (e.g., by light sources 412 which may have lenses 416 or filters) is effective to cause fluorescent molecules released, unquenched, or otherwise made detectable by the amplification reaction to fluoresce effective to allow detection of the amplified target nucleic acid molecules 410. Lines 414 indicate fluorescent radiation detectable by observation (e.g., through transparent or translucent upper surface 408). For example, fluorescent radiation may be detected, for example, with a detection assembly 308 as illustrated in FIG. 43C. As discussed above, such a detection assembly 308 includes an observational assembly 334 having optical elements such as, e.g., lenses and filters, for detection by eye or for use with a camera, photodetector, or other sensing device for electronic or by other detection.

Thus, as illustrated in FIGS. 48A, 48B and 48C, an initial step in the operation of a portion of an assay cartridge 306 comprises flow of nucleic acid molecules 394 from a processed sample 354 into assay chamber 320. A subsequent step is illustrated in FIG. 48B, showing assay chamber 320 in schematic side view after one or more thermal cycles, when amplification of target nucleic acid molecules 410 has occurred. A still later step in the operation of a portion of an assay cartridge 320 is illustrated in FIG. 48C, which shows assay chamber 320 in schematic side view, where illumination by light sources 412 induces fluorescence from fluorescent molecules released or otherwise made detectable by amplification of target nucleic acid molecules 410, allowing detection of the presence of target nucleic acid molecules 410 within a sample 354. As illustrated in these figures, an assay cartridge 306 may include multiple assay chambers 320; different probes and/or primers 404 directed to different target nucleic acid molecules may be present in different assay chambers 320, so that multiple target nucleic acid molecules may be tested for, and may be detected, from a single sample 354. In embodiments, the same probes and/or primers 404 directed to one target nucleic acid molecule may be provided within different assay chambers 320 to provide redundancy for greater assurance of accuracy. In embodiments, one or more assay chambers 320 may contain probes and/or primers 404 directed to a nucleic acid molecule normally found in a sample 354, so as to provide an internal control for comparison with amplification reactions in other assay chambers 320 on the same assay cartridge 306.

It will be understood that multiple target nucleic acid molecules may be detected with a single device. Different sets of probes and primers directed to different target nucleic acid molecules may be provided in different assay chambers 320, or different reaction chambers 186, or in different wells as discussed in the specification and the Examples above. In embodiments, different sets of probes and primers directed to different target nucleic acid molecules may be provided in a single assay chamber 320, or a single reaction chamber 186, or in a single well as discussed above. In such embodiments comprising different sets of probes and primers directed to different target nucleic acid molecules within a single chamber, well, or region, amplification of the different target nucleic acid molecules occurs at the same time within such single chambers, wells, or regions. Different target nucleic acid molecules may be detected and identified within a single chamber, well, or region, for example, where the probes and primers for different targets have different fluorescent molecules, allowing detection of each target by its distinctive identifying fluorescent signal.

Probes and/or primers 404 may be directed to target nucleic acid molecules found, for example, in pathogens, contaminants, or cancerous tissues. Pathogens whose presence may be tested for may be bacterial, viral, fungal, or other pathogens, and include, for example, tuberculosis, anthrax, streptococcus, staphylococcus, and other bacterial pathogens, viral, fungal and other pathogens, including other pathogens listed elsewhere in the specification.

It will be understood that the description of the methods, devices and systems herein is not meant to be limiting. Moreover, although individual features of the embodiments may be shown in some of the drawings and not in others, or described in conjunction with some devices and methods and not with others, those skilled in the art will recognize that individual features of one embodiment may be combined with any or all of the features of another embodiment.

1. A device for amplifying and/or detecting at least one nucleic acid molecule in a sample, comprising:
a first well region configured to receive probe molecules and primer molecules, said probe molecules and said primer molecules each configured to hybridize with at least a portion of a target nucleic acid molecule;
a second well region configured to receive target nucleic acid molecules; and
a connecting region providing a restricted pathway connecting said first well region and said second well region; said restricted pathway comprising an elongated channel configured to direct the flow of a liquid by capillary action and being effective to allow contact between said probe molecules, said primer molecules, and said target nucleic acid molecules so that at least one target nucleic acid molecule may be amplified and/or detected.

2. The device of claim 1, wherein said elongated channel has a width of less than about 2 mm and a depth of less than about 0.5 mm.

3. The device of claim 1, wherein said elongated channel has a width of about 0.3 mm and a depth of about 0.15 mm.

4. The device of claim 1, comprising a plurality of first well regions and a plurality of connecting regions.

5. The device of claim 1, wherein a connecting region comprises dried probe or primer molecules.

6. The device of claim 1, further comprising a valve connecting a first well region and a connecting region.

7. The device of claim 1, further comprising a divider configured to segment said connecting region, wherein said divider may be situated in place after said target nucleic acid molecules have been added to said second well region effective to separate said connecting region into a plurality of wells.

8. A device for amplifying and/or detecting at least one nucleic acid molecule in a sample, comprising:
a well having an interior region comprising a well base and configured to receive probe molecules, primer molecules, and target nucleic acid molecules, said probe molecules and said primer molecules each configured to hybridize with at least a portion of a target nucleic acid molecule;
said well base being transparent or translucent to radiation of at least a portion of the electromagnetic spectrum, having a plurality of base locations and comprising a thin gel layer having an area and configured to accept said probe, primer and target molecules effective to diffuse at least some of said probe, primer and target molecules into said thin gel layer to disperse said target molecules to different locations so that at a given location within the thin gel layer a target nucleic acid molecule may be contacted by probe molecules and primer molecules so that a target molecule may be amplified and/or detected at a given location.

9. The device of claim 8, comprising a plurality of wells.

10. The device of claim 8, wherein said target nucleic acid molecules disperse within said thin gel layer at a density of less than about 1 target nucleic acid molecules per square millimeter as viewed from above.

11. The device of claim 8, wherein said thin gel layer comprises a gel material selected from the group of gel materials consisting of gelatin, agar, agarose, acrylamide, Sepharose®, Sephadex®, Sephacryl®, casein, unfixed gels and cross-linked gels.

12. The device of claim 8, wherein said thin gel layer comprises a gel layer having a thickness of less than about 0.1 mm.

13. A device for amplifying and/or detecting at least one nucleic acid molecule in a sample, comprising:
a well having an interior volume comprising a gel and configured to receive probe molecules, primer molecules, and target nucleic acid molecules, said probe molecules and said primer molecules each configured to hybridize with at least a portion of a target nucleic acid molecule; and
a well base transparent or translucent to radiation of at least a portion of the electromagnetic spectrum;
said gel being configured so that at least some of said probe, primer and target molecules may diffuse into said gel to disperse said target molecules to different locations within said interior volume so that at a given location within the gel a target nucleic acid molecule may be contacted by probe molecules and primer molecules effective that a target molecule may be amplified and/or detected at a given location.

14. The device of claim 13, comprising a plurality of wells.

15. The device of claims 13, wherein said gel comprises a gel material selected from the group of gel materials consisting of gelatin, agar, agarose, acrylamide, Sepharose®, Sephadex®, Sephacryl®, casein, unfixed gels and cross-linked gels.

16. The device of claim 13, wherein said target nucleic acid molecules disperse into a volume at a density of less than about 10 target nucleic acid molecules per cubic millimeter.

17. A device for amplifying and/or detecting at least one nucleic acid molecule in a sample, comprising:
a plurality of hydrophilic wells separated from each other by a hydrophobic surface and each having an interior surface comprising probe molecules and/or primer molecules, said probe molecules and said primer molecules each configured to hybridize with at least a portion of a target nucleic acid molecule;
said plurality of wells being sized and spaced to receive target nucleic acid molecules for contact with probe molecules and primer molecules within a single well effective that a target nucleic acid molecule may be amplified and/or detected within a single well.

18. The device of claim 17, wherein said hydrophobic surface comprises an elongated post with an end, said end having a hydrophobic surface and having a plurality of depressions in said end comprising said plurality of hydrophilic wells.

19. The device of claim 18, wherein said depressions comprising said plurality of hydrophilic wells have depths of less than about 2 mm and widths of less than about 5 mm.

20. The device of claim 18, comprising a plurality of fiber optic bundles.

21. A device for amplifying and/or detecting at least one nucleic acid molecule in a sample, comprising:
a substrate having a substantially planar hydrophobic surface, a plurality of walls disposed on and substantially perpendicular to said surface, said walls defining a plurality of wells, said wells having a hydrophilic surface defining at least in part a volume configured to receive probe molecules, primer molecules, and target nucleic acid molecules, said probe molecules and said primer molecules each configured to hybridize with at least a portion of a target nucleic acid molecule effective that a target nucleic acid molecule may be amplified and/or detected within a depression.

22. The device of claim 21, wherein said wells comprise probe and/or primer molecules, each of said probe and/or primer molecules being configured to hybridize with at least a portion of a target nucleic acid molecule.

23. The device of claim 21, wherein said plurality of wells have depths of less than about 1 mm and widths of less than about 2 mm.

24. The device of claim 22, wherein said plurality of wells have depths of less than about 1 mm and widths of less than about 2 mm.

25. A method for amplifying and/or detecting at least one nucleic acid molecule in a sample, comprising:
contacting a first solution comprising a primer molecule and/or a probe molecule with a channel configured to conduct a solution along said channel, said probe molecules and said primer molecules each configured to hybridize with at least a portion of a target nucleic acid molecule;
drying said first solution effective that said primer and/or probe molecules is/are retained within said channel;
contacting a second solution comprising a target nucleic acid molecule with said channel configured to conduct a solution along said channel, effective to mix said target nucleic acid molecule, said dried probe and/or said primer molecules into said second solution within said channel at an initial temperature, wherein probes are cleaved by extending primers to generate fluorescence around targets that match the probes;
applying heat effective to raise the temperature of the mixed target nucleic acid, probe and primer molecules to a raised temperature above said initial temperature;
allowing the temperature to become reduced effective to reduce the temperature of the mixed target nucleic acid, probe and primer molecules from said raised temperature to a temperature closer to said initial temperature, said raising of temperature and allowing temperature to become reduced comprising a thermal cycle;
repeating said thermal cycle effective to produce nucleic acid copies of said target nucleic acid molecule effective to amplify and/or detect said target nucleic acid molecule.

26. The method of claim 25, wherein said thermal cycle is repeated after a short interval of time effective to prevent diffusion of said copies of said target nucleic acid molecules greater than a short distance from said individual target nucleic acid molecules, wherein said thermal cycles are repeated a number of times comprising no more than about twenty cycles.

27. The method of claim 25, wherein said conduction of solution along said channel comprises capillary action.

28. The method of claim 25, wherein said target nucleic acid molecule is detected by detection of an optical signal related to the presence of either said target nucleic acid molecule or of a copy of said target nucleic acid molecule.

29. The method of claim 25, wherein said contacting a solution with a channel comprises opening a valve.

30. A method for amplifying and/or detecting at least one nucleic acid molecule in a sample, comprising:
contacting a solution comprising a primer molecule and a probe molecule with a plurality of gel regions, said plurality of gel regions being disposed within a plurality of wells, each well comprising a gel region, said wells separated from other wells by barriers disposed between said wells, said probe and said primer molecules each configured to hybridize with at least a portion of a target nucleic acid molecule;
allowing at least some of said primer molecules and said probe molecules to diffuse into said gel;
contacting said gel with a second solution comprising a target nucleic acid molecule, effective to mix said target nucleic acid molecule with probe and primer molecules within said gel at an initial temperature, wherein said probes are cleaved by extending primers to generate fluorescence around targets that match the probes;
applying heat effective to raise the temperature of the mixed target nucleic acid, probe and primer molecules to a raised temperature above said initial temperature;
allowing the temperature to become reduced effective to reduce the temperature of the mixed target nucleic acid, probe and primer molecules from said raised temperature to a temperature closer to said initial temperature, said raising and allowing the temperature to become reduced comprising a thermal cycle;
repeating said thermal cycle effective to produce nucleic acid copies of said target nucleic acid molecule effective to amplify and/or detect said target nucleic acid molecule.

31. The method of claim 30, wherein said gel regions comprise thin gel layers having a thickness of less than about 0.1 mm.

32. The method of claim 30, wherein said thermal cycles are repeated after a short interval of time effective to prevent diffusion of said copies of said target nucleic acid molecules greater than a short distance from said individual target nucleic acid molecules, wherein the number of said thermal cycles comprises fewer than about twenty thermal cycles.

33. A method for amplifying and/or detecting at least one nucleic acid molecule in a sample, comprising:
placing a hydrophilic well in contact with a solution, said hydrophilic well comprising at least a portion of a device comprising a hydrophilic well surrounded by a hydrophobic surface, said solution comprising a target nucleic acid molecule, a primer molecule and a probe molecule, said probe molecules and said primer molecules each configured to hybridize with at least a portion of said target nucleic acid molecule;
removing said hydrophilic well from contact with said solution effective that a portion of said solution remains in contact with said hydrophilic well and substantially no solution remains in contact with said hydrophobic surface;
placing at least a portion of said device in contact with a hydrophobic liquid, said portion comprising at least a portion of said hydrophobic surface surrounding a well, effective that said solution in contact with said hydrophilic well remains in contact with said well;
applying heat effective to raise the temperature of the mixed target nucleic acid, probe and primer molecules from an initial temperature to a raised temperature above said initial temperature;
allowing the temperature to become reduced effective to reduce the temperature of the mixed target nucleic acid, probe and primer molecules from said raised temperature to a temperature closer to said initial temperature, said raising and said allowing the temperature to become reduced comprising a thermal cycle;
repeating said thermal cycle effective to produce nucleic acid copies of said target nucleic acid molecule effective to amplify said target nucleic acid molecule; and
detecting the presence of said target nucleic acid molecule or copies thereof.

34. A method for amplifying and/or detecting at least one nucleic acid molecule in a sample, comprising:
contacting a portion of a substrate comprising a hydrophilic well surrounded by a hydrophobic surface with a first solution, said first solution comprising a primer molecule and a probe molecule, said probe molecules and said primer molecules each configured to hybridize with at least a portion of a target nucleic acid molecule;
drying at least a portion of said substrate effective that said probe molecules and said primer molecules dry into at least a portion of said hydrophilic well;
contacting said hydrophilic well with a volume of a second solution comprising said target nucleic acid molecule;
removing said well from contact with said volume of said second solution, effective that a mixture of said target nucleic acid molecule, said probe and said primer remains in said second solution in contact with said well;
placing at least a portion of said substrate into contact with a hydrophobic liquid, effective that said mixture remains in contact with said well;
making copies of said target nucleic acid molecule within or adjacent said mixture; and
detecting the presence of said target nucleic acid molecule or copies thereof.

35. The method of claim 34, wherein said making copies comprises:
applying heat effective to raise the temperature of said mixture comprising target nucleic acid, probe and primer molecules from an initial temperature to a raised temperature above said initial temperature;
allowing the temperature to become reduced effective to reduce the temperature of said mixture from said raised temperature to a temperature closer to said initial temperature, said raising and said allowing the temperature to become reduced comprising a thermal cycle; and
repeating said thermal cycle effective to produce nucleic acid copies of said target nucleic acid molecule effective to amplify and/or detect said target nucleic acid molecule.

36. The method of claim 35, the method further comprising subjecting said mixture to a thermal cycle without substantial evaporation of fluid from said mixture.

37. A system for amplifying and/or detecting at least one nucleic acid molecule in a sample, comprising a plurality of assemblies, each of said assemblies configured for use with at least one other of said assemblies, comprising:
an optical assembly comprising an aperture and optical apparatus selected from a lens, a filter, and a window;
a reaction assembly comprising a reaction chamber configured for receiving a solution comprising a sample containing a target nucleic acid molecule and for reacting said sample with a primer molecule and a probe molecule in said reaction chamber; and
a thermal assembly configured to receive said reaction assembly, comprising a thermal cycling device capable of providing heat to said reaction assembly when said reaction assembly is received by said thermal assembly, and a controller configured for controlling the heat provided by said thermal cycling device.

38. The system of claim 37, wherein said reaction assembly further comprises a window, a support, a septum covering a port, and a filter separating said port from said reaction chamber effective to allow passage of a target nucleic acid molecule from said port to said reaction chamber while substantially preventing passage of cellular debris.

39. The system of claim 37, configured for use with a plurality of reaction assemblies, wherein said system is configured for use with a first reaction assembly or a second reaction assembly, and wherein said first reaction assembly may be replaced with said second reaction assembly.

40. The system of claim 37, wherein said reaction assembly comprises dried probe and/or primer molecules.

41. The system of claim 37, wherein said reaction assembly further comprises a plurality of electrodes effective for electrophoresing a target nucleic acid molecule upon provision of electrical power to said electrodes.

42. The system of claim 37, wherein said reaction assembly comprises a plurality of chambers.

43. The system of claim 41, wherein said reaction assembly comprises a plurality of chambers and wherein said plurality of electrodes is effective for electrophoresing a target nucleic acid molecule from one chamber to another chamber of said reaction assembly upon provision of electrical power to said electrodes.

44. The system of claim 37, wherein said reaction assembly further comprises a matrix effective to substantially prevent passage of cellular debris between chambers in said reaction assembly.

45. The system of claim 37, wherein said reaction assembly further comprises a matrix suitable for separation of cellular elements.

46. The system of claim 37, further comprising a light source.

47. The system of claim 37, further comprising a valve effective to regulate passage of material between chambers in said reaction assembly.

48. The system of claim 37, wherein said reaction assembly comprises dried probe or primer molecules.

49. The system of claim 43, wherein said reaction assembly comprises dried probe or primer molecules.

50. The system of claim 37, wherein said reaction assembly comprises a sample preparation module, at least one assay chamber configured for amplifying nucleic acid molecules therein, and a pathway configured to allow passage of fluid from said sample preparation module to said at least one assay chamber.

51. The system of claim 43, wherein said electrodes are configured to provide a first voltage gradient and a second voltage gradient, wherein said first voltage gradient has a first polarity, and said second voltage gradient has a second polarity, and wherein said first polarity and said second polarity are different.

52. The system of claim 51, wherein said first polarity of said first voltage gradient is perpendicular to said second polarity of said second voltage gradient.

53. The system of claim 51, wherein said electrodes are configured to provide said first voltage gradient at a different time than said second voltage gradient is provided.

54. The system of claim 51, wherein said reaction chamber comprises a matrix suitable for separation of cellular elements.
